(19) United States
(12) Patent Application Publication
(10) Pub. No.: US 2011/0287439 A1
(43) Pub. Date: Nov. 24, 2011

(54) CLASTOGENICITY TESTING

(75) Inventors: Annick Lauwers, Leuven - Heverlee (BE); Gerard Johan Griffioen, Leuven - Heverlee (BE); Hein Roger Duhamel, Leuven - Heverlee (BE); Nele Van Damme, Leuven - Heverlee (BE)

(73) Assignee: reMYND NV, Leuven - Heverlee (BE)

(21) Appl. No.: 13/058,337

(22) PCT Filed: Aug. 13, 2009
(86) PCT No.: PCT/EP2009/005877

§ 371 Date: May 25, 2011

(30) Foreign Application Priority Data
Aug. 14, 2008 (GB) 0814849.6

(51) Int. Cl. C12Q 1/68; C12Q 1/02; C12Q 1/66; C12N 1/19

(52) U.S. Cl. 435/6.13; 435/254.2; 435/29; 435/8

(57) ABSTRACT

The present invention relates to improved methods for detecting agents that cause or potentiate DNA damage and to genetically transformed cells that may be usefully employed in such methods.

The present invention relates to improved methods for detecting agents that damage DNA molecules and to genetically engineered cells that may be usefully employed in such methods.

### BACKGROUND OF THE INVENTION

DNA damage is induced by a variety of agents such as ultraviolet light, X rays, free radicals, methylating agents, topo-isomerase inhibitors, DNA synthesis inhibitors, reactive oxygen species generators and other mutagenic compounds. These agents may affect the integrity of genomic DNA caused by alterations, changes, rearrangements or damages to the DNA molecules in an organism including, but not limited to, mutations in genes or chromosomal rearrangements. In multicellular organisms these mutations can lead to carcinogenesis or in sexually propagating organisms may damage the gametes to give rise to congenital defects in offspring.

These DNA damaging agents may chemically modify the nucleotides that comprise DNA and may also break the phosphodiester bonds that link the nucleotides or disrupt association between bases (T-A or C-G). To counter the effect of these DNA damaging agents, cells have evolved a number of mechanisms. For instance the SOS response in E. coli is a well-characterized cellular response induced by DNA damage in which a series of proteins are expressed, including DNA repair enzymes, which repair the damaged DNA.

There are numerous circumstances when it is important to identify what agents may cause or potentiate alterations of DNA molecules. It is particularly important to detect agents that cause DNA damage when assessing whether it is safe to expose a person or animal to these agents. For instance a method of detecting these agents may be used as a genotoxicity assay for screening compounds that are candidate medicaments, food, food additives or cosmetics, to assess whether or not the compound of interest induces DNA damage.

Alternatively, methods for detecting DNA damaging agents may be used to monitor for contamination of water supplies and soil samples from suspected polluted sites with pollutants that contain mutagenic compounds.

Various methods, such as chromosome aberration tests, for determining the toxicity of an agent are known but are unsatisfactory for a number of reasons. For instance, incubation of samples can take many days when it is often desirable to obtain genotoxic data in a shorter time frame. Furthermore, many known methods for detecting DNA damage assay permanent DNA damage, as an endpoint, either in the form of misrepaired DNA (mutations and recombination's) or unrepaired damage in the form of fragmented DNA. However most DNA damage is repaired before such an endpoint can be measured and permanent DNA damage only occurs if the conditions are so severe that the repair mechanisms have been saturated. Changes associated with the process of DNA damage repair will therefore occur in a greater proportion of cells, and to a greater degree, than discernable genetic damage or other genetic endpoints.

Although lacking some metabolic pathways are having alternative comparative pathways to those found in animals and humans, basic DNA repair mechanisms, as a response to genetic damage, are similar between yeast and mammals.

The response to DNA damage in Saccharomyces cerevisiae (yeast) is well characterized. RAD54 encodes a structural element of the homologues recombination repair pathway (see below) and is transcriptional up-regulated in response to exposure of the yeast to a broad spectrum of genotoxins including, but not limited to, UV and X irradiation and alkylating agents and thus is a good surrogate for monitoring genetic endpoints (Cole et al. Molecular and Cellular Biology, 7: 1078-1084). RAD54 encodes a member of the DNA repair enzymes and is induced transcriptionally to above a constitutive level by a variety of different DNA lesions or damages, yet the promotor does not respond to non-genotoxic oxidative or reductive stresses, heat or osmotic shocks or amino acid starvation. In view of said characteristics, DNA damage can be monitored by the RAD54-induced transcription of a reporter protein, such as for example Green Fluorescent Protein (GFP) in the Greenscreen assay.

The Greenscreen genotoxicity test is disclosed in WO 98/44149, published on 8 Oct. 1998 (RAD54) and provides recombinant DNA molecules comprising a regulatory element, that activates gene expression in response to DNA damage, operatively linked to a DNA sequence that encodes a light emitting reporter protein. Such DNA molecules may be used to transform a cell and such cells may be used in a genotoxic test for detecting for the presence of an agent that causes or potentiates DNA damage. The cells may be subjected to an agent and the increased expression of the light emitting reporter protein from the cell, indicates that the agents cause DNA damage.

The genotoxicity tests described in WO 98/44149 detect the induction of DNA repair activity. The method described in WO 98/44149 may therefore be used to detect in a more specific way for the presence of DNA damaging agents.

WO 98/44149 further describes a number of useful genetic constructs that may be used to transform a host cell, in particular a yeast cell, such that it may be used in a genotoxic test. One such construct is yEGFP-444 (illustrated in FIG. 12 of WO 98/44149).

A number of mutant yeast strains now exist with altered phenotypes, including more permeable cell membranes and impaired or reduced export pump activity, that normally provide efficient detoxification of xenobiotics by deletion of their corresponding genes.

WO 05/12533, published on 10 Feb. 2005, describes a modified version of the Greenscreen assay comprising a spontaneous rearrangement of the vector described in WO 98/44149 which resulted in a brighter reporter.

A new protocol of the Greenscreen assay was developed by Knight et al. (Mutagenesis 22: 409-416, 2007) to enhance the metabolic competency of the yeast by the addition of rat liver S9 extract, with the aim of detecting genotoxicity from a greater number of promutagenic compounds.

Genetic analysis and subsequent biochemical characterization have defined three major DNA radiation damage repair pathways, namely the nucleotide excision repair pathway (NER), the recombination repair pathway and the postreplication repair and mutagenesis pathway (PRR). NER predominantly recognizes lesions that cause helical distortions, the recombination repair pathway is responsible for the repair of double strand breaks and PRR is defined as an activity to convert DNA damage-induced single-stranded gaps into large molecular weight DNA without actual removal of the replication-blocking lesions, which is often referred to as a DNA tolerance or avoidance pathway. In addition to the above identified three DNA radiation repair pathways, genes responsible for the repair of damaged bases belong to a base excision repair pathway (BER). The BER pathway recognizes and repairs specific base-modifying lesions that are relatively small modifications of the DNA predominantly produced by DNA alkylating agents and oxidative agents. There can be a strong interaction between BER and NER pathways. The sensitivity of eukaryotic genotoxicity testing systems may be further improved by inactivating certain DNA repair pathways (Jia et al. Sciences 75: 82-88, 2003).

All BER reactions are initiated by the action of a specific class of DNA enzymes called DNA glycosylases. A glycosylase recognizes and binds to the damaged site in a lesion specific manner and mediates the cleavages of the damaged base from the sugar backbone. MAG1 encodes a 3-methyladenine DNA glycosylase specifically involved in the repair of alkylated lesions. Mag1 has a broad range of substrates, including lesions produced by methylating and ethylating agents, as well as other industrial alkylating agents. The abasic site generated by Mag1 DNA glycosylase is further processed by apurinic/apyrimidinic(AP) endonucleases encoded by APN1 and APN2 in budding yeast.

There is a major need for accurate preregulatory screens and genetically engineered cells, that can help filter out genotoxins at an early stage of product development (e.g. drug development) when investment is relatively negligible.

The present invention detects induction of repair mechanisms in the BER, NER and recombination repair pathways. Of particular value is the demonstration that the present transformed cells and method can be used in high throughput screening of an actual pharmaceutical library of compounds, to detect compounds that are likely to be genotoxic in mammalian cells but missed by Ames, Vitotox or other prokaryotic-based screens. Through inactivation of repair pathways and through amplification of the induction of the repair signal of the beta-galactosidase reporter protein by the luciferin/luciferase reaction, the present invention is extremely sensitive for the detection of induction of repair and filters out clastogenic compounds in a more sensitive and predictive way as the Greenscreen assay.

### SUMMARY OF THE INVENTION

According to a first aspect of the invention, there is provided an eukaryotic cell comprising a regulatory element arranged to regulate expression of a reporter protein in response to DNA damage wherein said cell is characterized in that it is defective in a DNA repair pathway. The regulatory element can be present in a recombinant vector. The recombinant vector sequence can be integrated in the genome.

There is provided a cell that has an altered phenotype, including more permeable cell membranes and impaired export pumps that provide efficient detoxification by deletion of the corresponding genes. More specific said impaired export pumps can be Snq2 (SEQ ID No 4 and 5), Pdr5 (SEQ ID No 6 and 7) and Yor1 (SEQ ID No 8 and 9).

There is provided a cell wherein the defective DNA pathway is the excision repair (BER) pathway. The defect in the DNA repair pathway can be caused by the inactivation of MAG1 (SEQ ID No 10 and 11) and the regulatory element can comprises a yeast RAD54 promotor (SEQ ID No 1).

In another aspect of the invention, there is provided a cell, wherein the recombinant vector in addition to the regulatory element, comprises a DNA sequence that encodes a reporter protein. Said reporter protein can be an enzyme. More specifically said reporter enzyme can be the beta-galactosidase enzyme (SEQ ID No 2 and 3).

In another aspect of the invention there is provided a cell comprising the recombinant vector of FIG. 1 or a functional derivative thereof. Said cell can be a yeast cell such as Saccharomyces cerevisiae. More specific the Saccharomyces cerevisiae yeast cell is a SKAM4 cell, said SKAM4 cell;
having impaired activity of the export pumps Snq2, Pdr5 and Yor1;
having a defect in the BER DNA repair pathway caused by the inactivation of MAG1, and
comprising a recombinant vector sequence, wherein the RAD54 regulatory element is operatively linked to the beta-galactosidase gene.

The aforementioned SKAM4 cells have been deposited with the Belgian Coordinated collections of Microorganisms on Jul. 16, 2008 by ReMynd NV and received the accession number IHEM 22765.

In another aspect of the invention there is provided a method for preparing the SKAM4 cell, comprising the steps of:
deleting the export pumps,
making the RAD54-beta-galactosidase recombinant vector
integration of the RAD54-beta-galactosidase recombinant vector into the yeast genome, and
inactivating MAG1.

In yet another aspect of the invention there is provided a method of detecting the presence of an agent that causes or potentiates DNA damage, the method comprising subjecting a cell to an agent and monitoring gene expression wherein an increase of reporter gene expression indicates that the agent causes or potentiates DNA damage. More specifically there is provided a method of detecting the presence of an agent that causes or potentiates DNA damage, the method comprising subjecting a cell to an agent and monitoring the activity of the reporter protein, wherein an increase in reporter protein activity indicates that the agent causes or potentiates DNA damage. The method can comprise the following steps:
preparing yeast cells in the exponential growth phase,
adding the agent to be tested,
incubating the cells and
monitoring the expression of the reporter gene or the activity of the reporter protein.

The agent can be radiation, a free radical, a chemical, a biological, an environmental sample, a candidate medicament, a food additive or a cosmetic.

In another aspect of the invention, the reporter protein in the method is an enzyme more specifically the reporter protein is the beta-galactosidase enzyme.

In another aspect of the invention the activity of the reporter protein is measured by adding a lyses buffer containing a beta-galactosidase substrate to the cell culture, wherein the substrate is directly or indirectly converted to a luminescent product. In a particular embodiment the beta-galactosidase substrate consists of D-luciferin-o-B-galactopyranose (BetaGlo®), that can be cleaved by beta-galactosidase to luciferin and galactose. The luciferin can be used in a firefly luciferase reaction to generate light. The lyses buffer can contain both the beta-galactosidase substrate and the firefly luciferase and the lyses of the cells and the monitoring of the reporter protein activity can be performed in one step

In yet another aspect of the invention the method is further characterized in that it comprises the step of adding S9 extract to the cell culture prior to subjecting said cell to said agent. This liver extract allows to mimic metabolization of the test agent, and possible conversion into a DNA damaging agent by liver metabolic activity.

Another aspect of the invention provides a combination of a prokaryotic-based genotoxicity screening with a method as described herein.

Another aspect of the invention is concerned with the use of a cell of the invention in a method of identifying an agent that causes or potentiates DNA damage or with the use of a cell of the invention in a method of identifying a clastogen.

In a last aspect of the invention there is provided a kit comprising the cells of the invention.

### BRIEF DESCRIPTION OF THE DRAWING

FIG. 1: 212T(I)-pRAD54/betaGAL(FS-SK2) Plasmid

FIG. 2: Results in the Radarscreen with Methyl Methanesulfonate. For interpretation of graphs of reference compounds (FIG. 2-13) see Example 3f

FIG. 3: Results in the Radarscreen with Mitomycine C. Black: with S9, Grey: without S9.

FIG. 4: Results in the Radarscreen with 4-nitroquinoline-oxide. Black: with S9, Grey: without S9.

FIG. 5: Results in the Radarscreen with nalidixid acid. Black: with S9, Grey: without S9.

FIG. 6: Results in the Radarscreen with Benzo(a)pyrene. Black: with S9, Grey: without S9.

FIG. 7: Results in the Radarscreen with Ethidium Bromide. Black: with S9, Grey: without S9.

FIG. 8: Results in the Radarscreen with cis-platin. Black: with S9, Grey: without S9.

FIG. 9: Results in the Radarscreen with 2-aminofluorene. Black: with S9, Grey: without S9.

FIG. 10: Results in the Radarscreen with cyclophosphamide. Black: with S9, Grey: without S9.

FIG. 11: Results in the Radarscreen with 2-aminoanthracene. Black: with S9, Grey: without S9.

FIG. 12: Results in the Radarscreen with Methyl Viologen. Black: with S9, Grey: without S9.

FIG. 13: Results in the Radarscreen with Rifampicin. Black: with S9, Grey: without S9.

FIG. 14: Table 4 with results of the reference compounds in the Raderscreen assay. For interpretation of table 4 (FIG. 14) see Example 3f

### DETAILED DESCRIPTION

### Definitions

By “DNA damage” we mean any change, alteration or rearrangement of a DNA molecule in a cell.

By “DNA repair” or “DNA repair pathway” we mean a process or pathway present in order to restore the integrity of the DNA. Such process or pathway can be used to detect damage to DNA molecules in a cell.

By “clastogen” we mean an agent that causes breaks in chromosomes leading to sections of the chromosomes being deleted, added or rearranged.

By “carcinogen” we mean any agent involved in the promotion of cancer.

By “recombinant vector” we mean a DNA molecule with a selection marker gene that can be propagated in one or more host cells (e.g. E. coli and yeast) either as episomal plasmid or as integrated fragment in the genome and which may also carry additional DNA fragments that may be derived from different species that are not required for its propagation.

By “regulatory element” we mean a DNA sequence that regulates the transcription of a gene with which it can be associated.

By “operatively linked” we mean that the regulatory element is able to regulate the transcription of the reporter protein.

By “reporter gene” we mean a gene encoding a protein whose expression may be regulated by a regulatory element.

By “reporter protein” we mean a protein which is encoded by a reporter gene whose levels can be quantified by means of a suitable assay procedure.

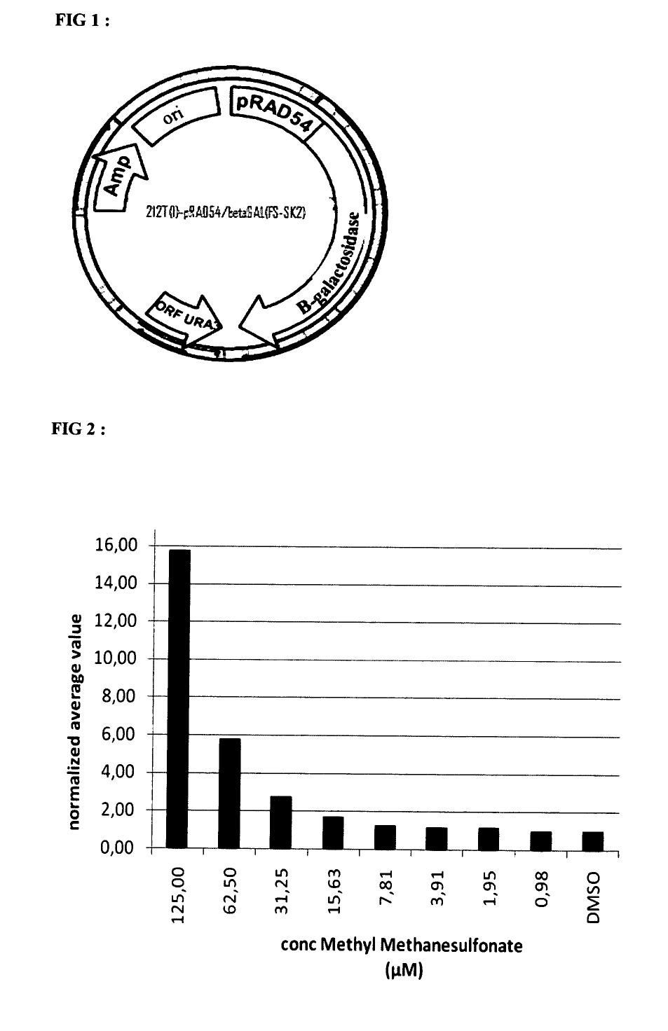

By “212T(I)-pRAD54/betaGAL(FS-SK2) pLASMID” we mean the recombinant vector illustrated in FIG. 1 of this specification which can be integrated in the yeast genome.

By “SKAM4” we mean the yeast strain constructed through genetically modifying the W303-1A strain (Thomas B. J. et at Cell 56: 619-630, 1989) by deleting genes encoding efflux pumps and a DNA repair gene and transformation and subsequent integration with a linearized recombinant vector containing the RAD54 promotor operatively linked to the beta-galactosidase gene; and deposited with the Belgian Coordinated collection of Microorganisms with the accession number IHEM 22765.

By “S9 extract” we mean a liver microsomal fraction containing the cytochromal P450 enzymes.

### Embodiments of the Invention

A method of the invention represents a novel cost-effective genotoxicity screen, that may be used to provide a pre-regulatory screening assay for use by the pharmaceutical industry and in other applications where significant numbers of compounds need to be tested. It provides a high throughput and a low compound consumption and is extremely sensitive to a broad spectrum of mutagens and importantly, clastogens.

The clastogenicity test of the invention is suitable for assessing whether or not an agent may cause DNA damage. It is particularly useful for detecting agents that cause DNA damage when assessing whether it is safe to expose a person to DNA damaging agents. For instance, the method may be used as an assay for screening whether or not known agents, such as radiation, free radicals, chemicals, biological, candidate medicaments, food additive or cosmetics, induce DNA damage. Alternatively, this method of the invention may be used to monitor for contamination of water supplies or polluted soils with pollutants containing DNA damaging agents.

The screening method of the invention may equally be used for assessing whether an agent may potentiate DNA damage. For example, certain agents can cause accumulation of DNA damage by inhibiting DNA repair (for instance by preventing expression or function of a repair protein) without directly inflicting DNA damage. These agents are often known as co-mutagens.

Accordingly in a first aspect of the invention, there is provided an eukaryotic cell comprising a regulatory element arranged to regulate expression of a reporter protein in response to DNA damage wherein said cell is characterized in that it is defective in a DNA repair pathway. In a particular embodiment the cell comprises a recombinant vector wherein the regulatory element is operationally lined to a reporter gene.

The vector backbone used in the cell of an aspect of the invention may comprise any suitable vector backbone known to those skilled in the art, which may be used to carry a reporter protein and a regulatory element. The recombinant vector of the present invention may for example be a plasmid, cosmid or viral vector. Such recombinant vectors are of great utility when replicating the DNA molecule. Furthermore, recombinant vectors are highly useful for transforming cells with the DNA.

The backbone may comprise a low copy number plasmid, a high copy number plasmid or an integrative vector. The backbone may be selected from the preferably well-known vectors YCplac22, YEplac112, YIplac204, Y1plac211, U1plac128, pRS303, pRS304, pRS305, pJW212T (BBA 1762 (2006) 312-318) or pRS306 (R. Daniel Gietz and Akio Sugino, New yeast-escherichia coli shuttle vectors constructed with in vitro mutagenized yeast genes lacking six-base pair restriction sites. Laboratory of Molecular Genetics. National institute of environmental health sciences, research triangle park, NC, 277709. Gene (1988)527-534). As provided in the examples hereinafter, in a particular embodiment the backbone consists of plasmid pJW212T.

The recombinant vectors are useful in the pharmaceutical industry for carrying out genotoxicity screens on novel compounds in the laboratory. It will be appreciated that, due to legislation involving use of genetically modified organisms, it is especially preferred that the vectors are only used in an enclosed environment, and not released in to the environment.

Recombinant vectors may be designed such that they may autonomously replicate in the nucleus of the cell. In this case, elements, which induce DNA replication, may be required in the recombinant vector. Hence, the vector may comprise an origin of replication, preferably, for yeast. Suitable origins of replication will be known to the skilled technician. For example, a suitable element derived from the yeast is the yeast 2 mm plasmid DNA replication origin or ARS (autonomiously replicating sequence) from yeast chromosomal DNA. Such replicating vectors can give rise to multiple copies of the DNA molecule in a transformant cell and are therefore useful when over-expression of the reporter protein is required. YCplac and YEplac vectors rely on an ARS or 2μ plasmid DNA replication origin in conjunction with a centromere sequence and are limited to one copy per cell. The transformant cell will be the cell according to the invention.

Instead of an autonomously replicating vector, the recombinant vector may be designed such that the vector and DNA molecule integrate into a chromosome of the host cell. Such integration has the advantage of improved stability compared to replicative plasmids. In this case, DNA sequences, which favor targeted integration (e.g. by homologous recombination) are desirable. For example, incorporation into the recombinant vector of fragments of the HO gene from chromosome IV of S. cerevisiae favors targeted integration in S. cerevisiae or cell lines derived there from. It may also be possible to integrate multiple copies of the integrating vector into the genome of the host cell. This will allow greater expression, and increase the signal output of the reporter protein even further.

The recombinant vector may comprise at least one selectable marker to enable selection of cells transfected with the vector, and preferably, to enable selection of cells harboring the recombinant vector that incorporates the DNA molecule of the first aspect. Examples of suitable selectable markers include genes conferring resistance to an antibiotic, for example, kanamycin, and ampicillin etc. Alternatively, or additionally, selectable markers may include auxotrophic markers, i.e. those which restore prototrophy, for example, yeast URA3, HIS3, TRP1 or LEU2 genes; in particular URA3.

The DNA sequence that encodes a reporter protein may code for any protein that can be quantified. However, it is preferred that the DNA sequence codes for an enzyme. The preferred DNA sequences that encode an enzyme is the gene for beta-galactosidase. As used herein the beta-galactosidase, consists of the Saccharomyces cereviseae enzyme encoded by the gene having the nucleic acid sequence represented by SEQ ID No 2, but is meant to include allelic variants as well as biologically active fragments thereof containing conservative or non-conservative changes as well as any nucleic acid molecule that is substantially identical, i.e. 70%, 75%, 80%, 85%, 87%, 89%, 90%, 92%, 93%, 94%, 95%, 96%, 97%, 98%, or 99% identical to the nucleic acid molecule encoding for the Saccharomyces cereviseae beta-galactosidase enzyme (SEQ ID No 2).

The aforementioned gene encodes for a beta-galactosidase enzyme having the amino acid sequence represented by SEQ ID No 3, but is meant to include allelic variants as well as biologically active fragments thereof containing conservative or non-conservative changes as well as artificial proteins that are substantially identical, i.e. 70%, 75%, 80%, 85%, 87%, 89%, 90%, 92%, 93%, 94%, 95%, 96%, 97%, 98%, or 99% identical to SEQ ID No 3.

Reporter activity of the reporter protein is measured by adding a beta-galactosidase substrate, to the cell culture. A specific substrate is D-luciferin-o-B-galactopyranoside, optionally further comprising luciferase that must be added in lyses buffer as it is not cell permeable. This substrate is cleaved by beta-galactosidase to luciferin and galactose. Luciferin is then used in a firefly luciferase reaction to generate light.

Beta-galactosidase may be used as a reporter protein because its measurement is simple and the enzymatic reporter protein in combination with the luciferase reaction causes an amplification of the DNA damage signal.

It is preferred that the regulatory element of the recombinant DNA molecule activates expression of a reporter protein when DNA damage occurs. Such regulatory elements ideally comprise a promoter sequence, which recruits RNA polymerase to the DNA vicinity of the start codon of the open reading frame and starts transcribing the DNA encoding the reporter protein. The regulatory element may also comprise other functional DNA sequences such as translation initiation sequences for ribosome binding or DNA sequences that bind transcription factors which promote gene expression following DNA damage. Regulatory elements may even code for proteins, which act to dislodge inhibitors of transcription from the regulated gene and thereby increase transcription of that gene.

Preferred regulatory elements are DNA sequences that are associated in nature with the regulation of the expression of DNA repair proteins. For instance, the regulatory elements from genes such as but not limited to RAD2, RAD6, RAD7, RAD18, RAD23, RAD51, RAD52, RAD54, CDC7, CDC8, CDC9, MAGI, PHR1, DINT, DDR48 and UB14 from yeast may be used to make recombinant DNA molecules present in the cells of the invention. Hence, the regulatory element used in the method of the invention may comprise genes such as RAD6, RAD7, RAD18, RAD23, RAD51, RAD52, RAD54, CDC7, CDC8, CDC9, MAG1, PHR1, DINT, DDR48 or 1JB14 from yeast.

A preferred regulatory element comprises the promoter and 5′ regulatory sequences of the RAD54 repair gene. Such a regulatory element may be derived from yeast and particularly from Saccharomyces cereviseae. The RAD54 gene as used herein, in particular consists of the Saccharomyces cereviseae RAD54 promotor having the nucleic acid sequence represented by SEQ ID No 1, but is meant to include allelic variants as well as biologically active fragments thereof containing conservative or non-conservative changes as well as any nucleic acid molecule that is substantially identical, i.e. 70%, 75%, 80%, 85%, 87%, 89%, 90%, 92%, 93%, 94%, 95%, 96%, 97%, 98%, or 99% identical to the nucleic acid molecule encoding for the Saccharomyces cereviseae RAD54 promotor (SEQ ID No 1).

Therefore, most preferred recombinant DNA molecules comprise a RAD54 regulatory element as defined herein operatively linked to a DNA sequence that encodes an enzyme. Accordingly, in one embodiment of the present invention, the preferred recombinant vector comprises the RAD54 gene operatively linked to the beta-galactosidase gene.

A preferred vector may comprise the RAD54 regulatory element, operatively linked to the beta-galactosidase gene and a nucleotide sequence adapted to integrate into the genome of a target cell.

Other DNA sequences, which favor targeted integration into the genome, and which may be incorporated into the recombinant vector include sequences from the ribosomal DNA array of S. cerevisiae. Such rDNA sequences favor targeted integration in to chromosome XII of S. cerevisiae or cell lines derived there from.

A preferred vector may therefore comprise the RAD54 regulatory element operatively linked to beta-galactosidase gene, and a nucleotide sequence adapted to integrate into the genome of a target cell, wherein the nucleotide sequence may be an rDNA sequence.

A preferred recombinant vectors is the 212T(I)-pRAD54/betaGAL(FS-SK2) plasmid shown in FIG. 1.

According to an aspect of the invention the recombinant vector is incorporated within a cell. Such host cells may be eukaryotic. Preferred host cells are yeast cells such as Saccharomyces cerevisiae. Yeast are preferred because they can be easily manipulated like bacteria but are eukaryotic and therefore have DNA repair systems that are more closely related to humans than those of bacteria.

Preferred yeast strains are constructed through genetically modifying the W303-1A strain (Thomas B. J. et al Cell 56: 619-630, 1989) by impairing efflux pumps and a DNA repair gene and transformation and subsequent integration with a linearized recombinant vector containing a regulatory element operatively linked to a reporter gene.

There is provided a cell that has an altered phenotype, including more permeable cell membranes and impaired export pumps that normally provide efficient detoxification, by deletion of the corresponding genes. More specific said impaired efflux pumps are selected from the group consisting of Snq2 (SEQ ID No 4 and 5), Pdr5 (SEQ ID No 6 and 7) and Yor1 (SEQ ID No 8 and 9). Accordingly in one aspect of the present invention the cell, i.e. yeast cell, with an altered phenotype is characterized in having at least one, in particular two or three impaired export mumps selected from Snq2 (SEQ ID No 4 and 5), Pdr5 (SEQ ID No 6 and 7) and Yor1 (SEQ ID No 8 and 9).

As used herein the nucleic acid sequences encoding the aforementioned efflux pumps, i.e. Snq2 (SEQ ID No 4), Pdr5 (SEQ ID No 6) and Yor1 (SEQ ID No 8) are meant to include allelic variants as well as biologically active fragments thereof containing conservative or non-conservative changes as well as any nucleic acid molecule that is substantially identical, i.e. 70%, 75%, 80%, 85%, 87%, 89%, 90%, 92%, 93%, 94%, 95%, 96%, 97%, 98%, or 99% identical to any one of the aforementioned export pump encoding polynucleotides.

By analogy, the aforementioned efflux pump proteins, i.e. Snq2 (SEQ ID No 5), Pdr5 (SEQ ID No 7) and Yor1 (SEQ ID No 9) are meant to include allelic variants as well as biologically active fragments thereof containing conservative or non-conservative changes as well as artificial proteins that are substantially identical, i.e. 70%, 75%, 80%, 85%, 87%, 89%, 90%, 92%, 93%, 94%, 95%, 96%, 97%, 98%, or 99% identical to any one of the aforementioned export pump proteins.

The impaired DNA repair pathway of the cells with an altered phenotype according to the present invention, can be anyone of the NER, the BER or the recombination repair pathway, and is typically realized by the inactivation of one or more genes in said DNA repair pathway.

The inactivated gene in the BER pathway may be the MAG1, the APN1 or the APN2 gene. The inactivated gene in the NER pathway may be the RAD2 gene. The inactivated gene in the recombinant repair pathway may be the RAD50 or the RAD52 gene.

In a preferred embodiment of the present invention the impaired DNA repair pathway in the cells with an altered phenotype of the present invention consist of the BER pathway or the NER pathway; more in particular the BER pathway. The preferred inactivated gene in the BER pathway is the MAG1 gene. The preferred gene in the NER pathway is the RAD2 gene. The MAG1 gene as used herein, in particular consists of the Saccharomyces cereviseae MAG1 gene having the nucleic acid sequence represented by SEQ ID No 10, but is meant to include allelic variants as well as biologically active fragments thereof containing conservative or non-conservative changes as well as any nucleic acid molecule that is substantially identical, i.e. 70%, 75%, 80%, 85%, 87%, 89%, 90%, 92%, 93%, 94%, 95%, 96%, 97%, 98%, or 99% identical to the nucleic acid molecule encoding for the Saccharomyces cereviseae RAD54 promotor (SEQ ID No 10).

By analogy, the MAG1 protein (SEQ ID No 11) is meant to include allelic variants as well as biologically active fragments thereof containing conservative or non-conservative changes as well as artificial proteins that are substantially identical, i.e. 70%, 75%, 80%, 85%, 87%, 89%, 90%, 92%, 93%, 94%, 95%, 96%, 97%, 98%, or 99% identical to Saccharomyces cereviseae MAG1 protein having SEQ ID No 11.

More preferred yeast strains are modified W303-1A strains;
having deleted efflux pumps,
having at least one inactivated DNA repair pathway,
comprising a linearized recombinant vector containing the RAD54 regulatory element operatively linked to a reporter gene.

The preferred yeast cell of the invention has an inactivation of the BER pathway.

The most preferred yeast cell is the SKAM4 cell;
having impaired activity of the export pumps Snq2, Pdr5 and Yor1;
having a defect in the BER DNA repair pathway caused by the inactivation of MAG1, and
comprising a recombinant vector sequence, wherein the RAD54 regulatory element is operatively linked to the beta-galactosidase gene.

According to an aspect of the invention, one of the DNA repair pathways of the transformed cell is inactivated. The inactivated DNA repair pathway may be the NER, the BER or the recombination repair pathway.

The preferred yeast cell of the invention has an inactivation of the BER pathway.

The inactivated gene in the BER pathway may be the MAG1, the APN1 or the APN2 gene. The inactivated gene in the NER pathway may be the RAD2 gene. The inactivated gene in the recombinant repair pathway may be the RAD50 or the RAD52 gene.

The preferred inactivated gene in the BER pathway is the MAG1 gene. The preferred gene in the NER pathway is the RAD2 gene.

The inactivated gene for the aforementioned efflux pumps or within the aforementioned DNA repair pathways may be mutated or deleted or its corresponding mRNA levels are reduced. The inactivated protein may be non-functional or not-expressed.

Inactivation through down-regulation of expression can include, but is not limited to for example, antisense RNA molecules, ribozymes and small interfering RNA (RNAi) molecules.

Antisense nucleic acid molecules within the invention are those that specifically hybridize (for example bind) under cellular conditions to cellular mRNA and/or genomic DNA encoding a DNA repair protein in a manner that inhibits expression of the DNA repair protein, for example, by inhibiting transcription and/or translation. The binding may be by conventional base pair complementarily, or, for example, in the case of binding to DNA duplexes, through specific interactions in the major groove of the double helix. Methods for design of antisense molecules are well known to those of skill in the art. General approaches to constructing oligomers useful in antisense therapy have been reviewed, for example, by Van der Krol et al. (1988) Biotechniques 6:958-976; Stein et al. (1988) Cancer Res 48:2659-2668; and Narayanan, R. and Aktar, S. (1996): Antisense therapy. Curr. Opin. Oncol. 8(6):509-15. As non-limiting examples, antisense oligonucleotides may be targeted to hybridize to the following regions: mRNA cap region; translation initiation site; translational termination site; transcription initiation site; transcription termination site; polyadenylation signal; 3′ untranslated region; 5′ untranslated region; 5′ coding region; mid coding region; and 3′ coding region.

An antisense construct can be delivered, for example, as an expression plasmid which when transcribed in the cell produces RNA which is complementary to at least a unique portion of the cellular mRNA which encodes a repair gene product. Alternatively, the antisense construct can take the form of an oligonucleotide probe generated ex vivo which, when introduced into a repair gene expressing cell, causes selective inhibition of expression of the corresponding gene by hybridizing with an mRNA and/or genomic sequence coding for the repair gene. Such oligonucleotide probes are preferably modified oligonucleotides that are resistant to endogenous nucleases, for example exonucleases and/or endonucleases, and are therefore stable in vivo. With respect to antisense DNA, oligodeoxyribonucleotides derived from the translation initiation site, for example, between the −10 and +10 regions of a repair gene encoding nucleotide sequence, are preferred.

The antisense molecules can be delivered into cells that express the repair gene in vivo. A number of methods have been developed for delivering antisense DNA or RNA into cells and are well known in the art. Because it is often difficult to achieve intracellular concentrations of the antisense sufficient to suppress translation of endogenous mRNAs, a preferred approach utilizes a recombinant DNA construct in which the antisense oligonucleotide is placed under the control of a strong promoter. The use of such a construct to transfect target cells in a subject preferably will result in the transcription of single-stranded RNAs that will hybridize with endogenous transcripts encoding the gene products of interest in sufficient amounts to prevent translation of the respective mRNAs. For example, a vector can be introduced in vivo such that it is taken up by a cell and directs the transcription of an antisense RNA. Such a vector can remain episomal or can become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells.

Expression of the sequence encoding the antisense RNA can be by any promoter known in the art to act in mammalian, and preferably human cells. Such promoters can be inducible or constitutive. Such promoters can include, but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), and the regulatory sequences of the metallothionein gene (Brinster et al., 1982, Nature 296:39-42).

A ribozyme can also down-regulate expression of a repair gene product. Ribozyme molecules are designed to catalytically cleave a transcript of a gene of interest, preventing its translation into a polypeptide. (See, for example, Sarver et al. (1990) Science 247:1222-1225 and U.S. Pat. No. 5,093,246). In general, ribozymes catalyze site-specific cleavage or ligation of phosphodiester bonds in RNA. While various forms of ribozymes that cleave mRNA at site-specific recognition sequences can be used to destroy repair gene mRNAs, the use of hammerhead ribozymes is preferred. Hammerhead and hairpin ribozymes are RNA molecules that act by base pairing with complementary RNA target sequences, and carrying out cleavage reactions at particular sites. In the case of the hammerhead, the ribozyme cleaves after UX dinucleotides, where X can be any ribonucleotide except guanosine, although the rate of cleavage is highest if X is cytosine. The catalytic efficiency is further affected by the nucleotide preceding the uridine. In practice, NUX triplets (typically GUC, CUC or UUC) are required in the target mRNA. Such targets are used to design an antisense RNA of approximately 12 or 13 nucleotides surrounding that site, but skipping the C, which does not form a conventional base pair with the ribozyme.

Synthetic hammerhead ribozymes can be engineered to selectively bind and cleave a complementary mRNA molecule, then release the fragments, repeating the process with the efficiency of a protein enzyme. This can represent a significant advantage over, for example, antisense oligonucleotides which are not catalytic, but rather are stoichiometric, forming a 1:1 complex with target sequences. The hammerhead ribozymes of the invention can be designed in a 6-4-5 stem-loop-stem configuration, or any other configuration suitable for the purpose. In general, because the chemical cleavage step is rapid and the release step is rate-limiting, speed and specificity are enhanced if the hybridizing “arms” of the ribozyme (helices I and III) are relatively short, for example, about 5 or 6 nucleotides. Suitability of the design of a particular configuration can be determined empirically, using various assays known to those of skill in the art.

Antisense RNA and ribozyme molecules of the invention may be prepared by any method known in the art for the synthesis of such molecules. These include techniques for chemically synthesizing oligodeoxyribonucleotides and oligoribonucleotides well known in the art, such as for example solid phase phosphoramide chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters. Alternatively, antisense cDNA constructs that synthesize antisense RNA constitutively or inducible, depending on the promoter used, can be used.

In a deletion or knockout, the target gene expression is undetectable or insignificant. A knock-out of a repair gene means that functional expression of the repair gene has been substantially decreased so that the repair protein expression is not detectable or present at reduced levels. This may be achieved by a variety of mechanisms, including introduction of a disruption of the coding sequence, e.g. insertion of one or more stop codons, insertion of a DNA fragment, etc., deletion or partial deletion of the coding sequence, substitution of stop codons for coding sequence, etc. In some cases the exogenous transgene sequences are ultimately deleted from the genome, leaving a net change to the native sequence. Different approaches may be used to achieve the “knock-out”. A chromosomal deletion of all or part of the native gene may be induced, including deletions of the non-coding regions, particularly the promoter region, 3′ regulatory sequences, enhancers, or deletions of gene that activate expression of repair genes. A functional knock-out may also be achieved by the introduction of an anti-sense construct that blocks expression of the native genes (for example, see Li and Cohen (1996) Cell 85:319-329). “Knock-outs” also include conditional knock-outs, for example where alteration of the target gene occurs upon exposure of the cell to a substance that promotes target gene alteration, introduction of an enzyme that promotes recombination at the target gene site (e.g. Cre in the Cre-lox system), or other methods known in the art.

DNA constructs for homologous recombination will comprise at least a portion of the repair gene with the desired genetic modification, and will include regions of homology to the target locus. Methods for generating cells having targeted gene modifications through homologous recombination are known in the art. For various techniques for transfecting eukaryotic cells, see Keown et al. (1990) Methods in Enzymology 185:527-537.

Another yeast cell of the invention may have more than one inactivated repair gen.

There is provided a cell that has an altered phenotype, including more permeable cell membranes and impaired export pumps that provide efficient detoxification by deletion of the corresponding genes. More specific said impaired export pumps can be Snq2, Pdr5 and Yor1.

According to an aspect of the invention the transformed cell can have an altered phenotype, including more permeable cell membranes and/or cell walls and impaired export pumps that provide efficient detoxification. As described herein above this altered phenotype can be obtained by antisense RNA molecules, ribozymes, deletions through homologues recombination or through inhibitors. A gene involved in cell wall integrity is for example ERG6. In a preferred embodiment the impaired export pumps are Snq2, Pdr5 and Yor1.

Host cells used for expression of the protein encoded by the DNA molecule are ideally stably transformed, although the use of unstably transformed (transient) cells is not precluded.

In another aspect of the invention there is provided a method for preparing the SKAM4 cell, comprising the steps of:
deleting the export pumps,
making the RAD54-beta-galactosidase recombinant vector
integration of the RAD54-beta-galactosidase recombinant vector into the yeast genome, and
inactivating MAG1.

Genetically engineered cells according to an aspect of the invention may be prepared by the procedures described in the examples. The cell according to one aspect of the invention are ideally unicellular organisms such as yeast (for instance one of the strains described above).

The method of the invention may comprise subjecting a cell, such as the SKAM4 yeast cell, to an agent and monitoring gene expression wherein an increase of reporter gene expression indicates that the agent causes or potentiates DNA damage. The method of the invention may comprise subjecting a cell, such as the SKAM4 yeast cell, to an agent and monitoring the activity of the reporter protein, wherein an increase in reporter protein activity indicates that the agent causes or potentiates DNA damage.

Such genetically engineered cells may be used according to the method of the invention to assess whether or not agents induce or potentiate DNA damage. Beta galactosidase expression is induced in response to DNA damage. A substrate is cleaved by beta-galactosidase. Depending on the substrate used and possible additional reactions, the final reaction product can be measured by a suitable means, such as but not limited to a colorimeter, a fluorometer or a luminometer, as an index of the DNA damage caused.

The response to DNA damage, may be evaluated either in a suspension of a defined number of whole cells or from a defined amount of material released from cells following breakage. The method of one aspect of the invention is particularly useful for detecting agents that induce DNA damage at low concentrations. The methods may be used to screen agents such as radiation, free radicals, chemicals, biologicals, environmental samples, candidate medicaments, food additives or a cosmetics to assess whether it is safe to expose a living organism, particularly people, to such agents.

The method of the invention may be employed to detect whether or not water supplies or soil samples from suspected polluted sites, are contaminated by DNA damaging agents or agents that potentiate DNA damage. For instance, the methods may be used to monitor industrial effluents for the presence of pollutants that may lead to increased DNA damage in people or other organisms exposed to the pollution.

The method of the invention may comprise the following steps:
preparing yeast cells in the exponential growth phase,
adding the agent to be tested,
incubating the cells and
monitoring the expression of the reporter gene or the activity of the reporter protein.

The reporter protein in the method of the invention can be an enzyme, most preferably the enzyme is the beta-galactosidase enzyme.

The activity of the reporter protein can be measured by adding a lysis buffer containing a beta-galactosidase substrate to the cell culture, wherein the substrate is directly or indirectly converted to a detectable product such as but not limited to a coloured product, a chemiluminescent product or a fluorogenic product.

The beta-galactosidase substrate can be cleaved by beta-galactosidase to for example luciferin and galactose wherein luciferin is then used in a firefly luciferase reaction to generate light.

In the method of the invention, the lyses buffer can contain both the beta-galactosidase substrate and the firefly luciferase and lyses of the cells and monitoring the reporter protein activity can be performed in one step.

The yeast cell in the exponential growth preferably have a density of between 1-3 when starting the experiment. Different types of multi-well plates can be used, preferably 96-well plates. Agent can be added at different concentration. Incubation of the multi-well plate can be performed between 1-12 hour, preferably for 6 hours. Incubation temperature should be between 20 and 37 degrees Celsius, preferably at 30 degrees Celsius.

When appropriate different lyses buffers can be used, preferably the Beta-Glo® cell lyses buffer. The Beta-Glo® cell lyses buffer contains the beta-galactosidase substrate and all the required factors for the luciferase reaction.

Different beta-galactosidase substrates can be added to the lyses buffer such as but not limited to:
Galacton®, Galacton-start® or Galacton-plus®
Fluorescein di-β-D-galactopyranoside, resorufin β-D-galactopyranoside, Methylumbelliferyl-Beta-D-galactopyranoside, 4-Methylumbelliferyl-β-D-galactopyranoside, Fluorescein di(beta-D-galactopyranoside)
o-Nitrophenyl-β-D-galactopyranoside, chlorofenol red-β-D-galactopyranoside or D-luciferin-o-β-galactopyranoside. Preferably the substrate is D-luciferin-o- β-galactopyranoside.

At the start and the end of the incubation the amount of cells is determined spectrophotometrical by measuring turbidity at 595 nm. After adding for example, a substrate, production of light is measured with a luminescence reader. The luminescence signal is corrected for the cell density. A normalized luminescence value is calculated dividing the luminescence value of the sample by the luminescence value of a reference.

Agents may be metabolized in the method of the invention. Metabolization of the tested agent may be accomplished by expressing the respective mammalian enzymes in the yeast reporter strain. Alternatively hepatic mammalian enzymes present in the post mitochandrial S9 extract, comprising the microsomes can be added to the method of the invention.

Thus the method as described above can be performed with an additional step wherein S9 extract is added to the cells before addition of the agents to be tested. The S9 extract is a liver microsomal fraction containing the cytochromal P450 enzymes.

Hence, the method of the invention can be further characterized in that it comprises the step of adding S9 extract to the cell culture prior to subjecting said cells to said agent.

Prokaryotic-based genotoxicity screenings, such as for example the Vitotox™ are very sensitive for the detection of mutagenic compounds. The present invention is very sensitive in detecting clastogenic and carcinogenic compounds. When a prokaryotic-based genotoxicity screening is combined with the present invention, the whole range of DNA damaging agents will be detected in a very sensitive way.

A further aspect of the present invention is thus a combination of a prokaryotic-based genotoxicity screening with the method of the invention.

The present invention also encompass the use of the cells of the invention in a method of identifying an agent that causes or potentiates DNA damage and more in particular the use of a cell in a method of identifying a clastogen.

The agents and cells described herein can be packaged as a kit. Thus, one or more agents can be present in a first container, and the kit can optionally include one or more agents in a second container. The kit can include instructions describing the method of the present invention. The agents, cells, containers and/or the instructions can be present in a package.

The contents of the kit can contain but is not limited to the frozen cells, the growth medium, buffers, multiwell plates, S9 extract+cofactors, enzyme substrates, reference compounds etc.

### EXPERIMENTAL PART

### Example 1

### Production of S9 Extract

S9 extracts are prepared from adult male Wistar rats. The rats were injected intraperitonially with a solution (20% w/v) of Aroclor 1254 (500 mg/kg body weight) in corn oil. Five days later, the rats were killed by decapitation. The livers were minced in a blender and homogenized in 3 volumes of phosphate buffer with a potter homogenizer. The homogenate was centrifuged for 15 min. at 9000 g. The supernatant (S9 fraction) was transferred into sterile ampules, which were stored in liquid nitrogen.

### Example 2

### Construction of the SKAM4 Strain

Yeast strain SKAM4 has the beta-galactosidase gene (which is under transcriptional control by the RAD54 promoter) integrated at the URA3 locus. In addition genes encoding efflux pumps (Snq2, Pdr5 and Yor1) and a gene encoding a protein involved in DNA repair (Mag1) are deleted.

### Example 2a

### Construction of SPY Strain

Strain W303-1A (REF: B. J. Thomas, R. Rothstein, Elevated recombination rates in transcriptional active DNA, Cell 56 (1989) 619-630) was used for deletion of SNQ2 by homologous recombination. A PCR fragment was generated using primers SNQ2F and SNQ2R and genomic DNA of a yeast snq2::KanMX deletion strain (Euroscarf Acc. No. Y03951) as template. This fragment was transformed to W303-1A cells and transformants were selected on suitable solid growth medium containing antibiotic G418. This resulted in strain W3_dS.

Next A PCR fragment was generated using primers PDR5F1 and PDR5R and an appropriate plasmid-borne HIS3 gene (Saccharomyces cerevisiae) as template. This fragment was transformed to strain W3_dS and transformants were selected on suitable solid growth medium lacking histidine. This resulted in strain W3_dSP. Next A PCR fragment was generated using primers YORF1 and a YOR1R an appropriate plasmid-borne TRP1 gene (Saccharomyces cerevisiae) as template. This fragment was transformed to strain W3_dSP and transformants were selected on suitable solid growth medium lacking tryptophane. This resulted in strain W3_dSPY.

### Example 2b

### Construction of Plasmid 212T(I)-pRAD54/betaGAL(FS-SK2)

Plasmid pJW212T (BBA 1762 (2006) 312-318) was cut with XbaI and the vector backbone was ligated to a circular plasmid named pXY212T(I). Subsequently, a DNA fragment containing the beta-galactosidase gene was cloned (using restriction enzymes BamHI and StuI) into pXY212T(I) resulting in plasmid 212T(I)-LacZ. A DNA fragment containing the RAD54 promoter was generated by PCR using primers RAD54P_F and RAD54P_R and yeast (Saccharomyces cerevisiae) genomic DNA as template and was cloned (using restriction enzymes BamHI and NgoMVI) in plasmid 212T(I)-LacZ. This resulted in plasmid 212T(I)-pRAD54/betaGAL(FS-SK2) containing a functional fusion of the RAD54 promoter with the beta-galatosidase gene.

### Example 2c

### Integration Plasmid 212T(I)-pRAD54/betaGAL(FS-SK2) in W3 dSPY

Plasmid 212T(I)-pRAD54/betaGAL(FS-SK2 was cut with SbfI and subsequently transformed to strain W3 dSPY. Transformants were selected on suitable solid growth medium lacking uracil. This resulted in strain SK2A3.

### Example 2d

### Deletion of Mag1 in Strain SK2A3

Next A PCR fragment was generated using primers MAG1/Leu2-F1 and MAG1/Leu2-R1 and an appropriate plasmid-borne LEU2 gene (Saccharomyces cerevisiae) as template. This fragment was transformed to SK2A3 cells and transformants were selected on suitable solid growth medium lacking leucine. This resulted in strain SKAM4.

Genotype SKAM4 MATa leu2-3/112 ura3-1 trp1-92 his3-11, 15 ade2-1 can1-100 snq2::kanMX4 pdr5::HIS3 yor1::TRP1 ura3:: URA3/pRAD54-betaGAL mag1::LEU2.

### Primer Sequences Used in Example 2a, 2b and 2d:

SNQ2F
5′-CCGCCCATTTCCGTTTAAATCCG-3′
SNQ2R
5′-TTTTCCTGTGTCCAATTTTTTTATTTTC-3′
YORF1
5′-AAAAGATTAATATTACTGTTTTTATATTCAAAAAGAGTAAAGCCG
TTGCTATATACGAATCAGATTTTATGTTTAGATCTTTTATGCTT-3′
YOR1R
5′-GTACCATCGGCAACATATAAATAAATAAAAGAGAAAAATCATGCA
ACAAATAATATAAATGAGGGCCAAGAGGGAGGGCAT-3′
PDR5F1
5′-AAGAAATTAAAGACCCTTTTAAGTTTTCGTATCCGCTCGTTCGAA
AGACTTTAGACAAAAGAGTGCACCATAATTCCGTTTTAAGA-3′
PDR5R
5′-ATGTTTATTAAAAAAGTCCATCTTGGTAAGTTTCTTTTCTTAACC
AAATTCAAAATTCTATTTCCTGATGCGGTATTTTCTCCTT-3′
RAD54P_F
5′-TGGTACCGGGCCGGCTGCGCTACGGTTCCTGCCGCTC-3′
RAD54P_R
5′-GTACCCGGGGATCCATGCATCAGTTATAAGGAAATATATATGGTA
CC-3′
MAG1/Leu2-F1
5′-TAAGTTATCTATGAATCAATGAGAATTGGCCACTGCCCTCTGATA
TGACGATGGAAGTGGGCGCACATTTCCCCGAAAAGTGCCACCTGACGT
C-3′
MAG1/Leu2-R1
5′-CCCTACGAGAAGCTGTAAATATGAATTTCTTTAGTAGGCATCACA
CACAA CAATAGGGTGGGTCCGGTTAAACGGATCTCGCATTGATGAGG
CAAC

### Example 3

### Radarscreen with Luminescence and S9 Metabolic Activation

### Example 3a

### Principle

Yeast strain SKAM4 bears a RAD54-LacZ reporter construct, which is responsive to agents that affect the integrity of its genomic DNA. Compounds supplied to the growth medium can be evaluated for genotoxicity by determining their effect on reporter gene expression. Reporter activity is measured by adding Beta-Glo® cell lyses buffer, containing a beta-galactosidase substrate, to the yeast culture. The substrate is cleaved by beta-galactosidase to luciferin and galactose. Luciferin is then used in a luciferase reaction to generate light. Metabolic activation of the compounds is obtained by adding post mitochondrial supernatant S9.

### Example 3b

### Equipment and Products

### Equipment:

Microplate reader: Multiskan Ascent: model 354 (ThermoLabsystems)

Microplate shaker: Titramax 101 (Heidolph Instruments)

Microtiterplate lids: polystyreen, sterile (Greiner: #656161)

Microplates96 well polystyrene cell culture μClear WHITE (Greiner: #655 098)

Microplate reader luminescence: Infinite M200 Tecan

### Products:

BAP (benzo[A]pyrene) 2 μl BAP (500 μg/ml) per well is added when appropriate

Beta-Glo® Assay System cat nr. E4740 (promega) 100 ml

The lysis buffer is stored in 10 ml portions in −20° C.

Post mitochondrial S9 extract Trinova Biochem (Moltox of Notox) (#11-101.8) 8 ml. The extract is stored at −80° C. in portions of 400 μl

Glucose-6-phosphate Sigma (# G6526)-1 g

NADP Sigma (#N5755-250MG)

KPO4 Sigma (#P3786-100G)

MgCl2 hexa-hydrate VWR (#1.05833.0250)

### Yeast Growth Medium: SC-URA (Liquid/Solid)

The following components are dissolved in distilled water
0.77 g/l CSM-URA (MP biomedicals #4511-222)
5 g/l ammoniumsulfate for biochemistry (Fw: 132,14) (Merck: # 1.01211.1000)
1.7 g/l yeast nitrogen base W/O ammonium sulphate & amino acid (Remel: #459932; distributed by Oxoid)
0.05 g/l adenine (6-aminopurine, Fw: 135,1) (MP biomedicals #4060-012)

The solution is autoclaved at 120° C. for 20 min. 2% D(+)-glucose-monohydrate (50 ml/l from a 40% autoclaved solution in distilled water) (Merck: #1.08342.2500, Fw: 196,17) is added. Alternatively D(+)-glucose-monohydrate is added immediately and the solution is filtersterilized. The medium is stored at room temperature.

In case of solid medium 2% agar (Oxoid: #LP0011) is added to the solution before sterilization and the pH is adjusted to 6.5 with 4M NaOH.

### Example 3c

### Method

A plate culture is prepared with the stock of SKAM4 cells from −80° C. This plate can be stored for +/−2 months.

A liquid stock culture of strain SKAM4 is made by inoculating a small amount of cells from the plate culture in 50 ml medium in a 100 ml erlenmeyer and grown at 30° C., 200 rpm overnight. This culture is to be stored at 4° C. and can be used at least 1 to 2 weeks for subsequent experiments.

### Day 1

5, 10, 20, 40, 80 μl from the stock culture is inoculated in 50 ml medium and grown overnight at 30° C., 200 rpm. The aim is to obtain the next day at least 1 culture with an OD₅₉₅ of about 1-3.

### Day 2

S9-mixture with and without S9 is prepared. The following quantities gives the ml required for 1 microtiter plate:

| | Buffer | Buffer with S9 |
|---|---|---|
| S9 extract | 0 | 140 |
| KCl/MgCl₂ | | |
| (0.1M/0.1M) | 10 | 10 |
| NADP⁺ (26 mM) | 43 | 43 |
| glucose-6-P (66 mM) | 80 | 80 |
| KPO₄ (200 mM) | 266 | 266 |
| H₂O | 301 | 161 |
| Total | 700 | 700 |

Each plate is vortexed before measuring OD₅₉₅.

OD₅₉₅ of the overnight cultures has to be determined. The culture with OD₅₉₅ closest to 2 is diluted to OD₅₉₅˜0.5 with growth medium.

600 μl S9-mixture is added to 5280 μl of the diluted culture and 600 μl buffer without S9 to 5280 μl of the diluted culture (these are quantities to fill 1 plate). The cells must be homogeneously in suspension (for instance by stirring the reservoir containing the cell suspension). 98 μl/well is dispensed in 96 well assay plates (Microplates96 well polystyrene cell culture μClear WHITE). This leads to a final concentration of 2% S9 in the assay. Medium is added in the wells of column12 to correct for background OD. The OD₅₉₅ is measured after the plates have been vortexed rigorously to resuspend the precipitated yeast cells.

Compounds (2 μl) are added to each well (maximum final concentration of DMSO should not exceed final concentration of 2% w/v). Each measurement is performed at least in triplicate for every compound. Column 11 is used for the control DMSO (2%). An exemplary plate layout is shown below. In this particular lay-out every compound is assayed in triplicate with and without S9. Row 1 and row 8 can be used for the control (BAP).

The OD₅₉₅ is measured after the plates have been vortexed rigorously to resuspend the precipitated yeast cells.

The assay plates are incubated for 6 hours at 30° C.

The OD₅₉₅ is measured after plates have been vortexed rigorously to resuspend the precipitated yeast cells.

50 μl Beta-Glo® cell lyses buffer is added to the wells and the plate is vortexed for 45-60 min whereafter luminescence is measured.

### Example 3d

### Data Handling

Calculation of the amount of cells by correcting and normalizing OD595 nm is performed with the following formula:

“OD595 after incubation—OD595 compound—OD595 medium without yeast cells”

Wells with 50% or more reduction in yeast cell growth in comparison with the wells of a control vehicle (DMSO) are not included.

Luminescence read out blanc correction is performed by subtraction of the luminescence value of medium without yeast cells from the luminescence read out of a sample with compound and dividing it by the corrected and normalized OD595 value. Then the normalized luminescence value is calculated by dividing the corrected luminescence values by the corrected value of a control vehicle (DMSO) (e.g. average value compound/average value DMSO). A compound is considered genotoxic when the normalized value is 1.5 or higher

Example lay-out of assay plate (10 cmpds, 3×). BAP is the control to check whether cells are responsive to known mutagens. Vehicle (usually DMSO) is the negative control.

### Example 3f

### Results

### Definitions

Sensitivity=number of correctly identified positives/number of correctly identified positives+number of false negatives
Specificity=number of correctly identified negatives/(number of correctly identified negatives+number of false positives)
Predictivity=(number of correctly identified positives+number of correctly identified negatives)/total number of tested compounds
n=number of tested compounds
correctly identified means: compared with results from the Ames test (for mutagenicity) or with in-vitro data for clastogenicity/carcinogenicity.
   Reference Vitotox assay:

Luc Regniers, Brigitte Borremans, Ann Provoost and Luc Verschaeve. The VITOTOX® test, an SOS bioluminescence Salmonella typhimurium test to measure genotoxicity kinetics. Daniël van der Lelie*, Environment Division, Flemish Institute for Technological Research (VITO), Boeretang 200, B2400 Mol, Belgium. Mutation Research 389 (1997)279-290.

### Reference Greenscreen Assay:

Hastwell P. W. et al (2006)

Validation of the GreenScreen HC GADD45a-GFP genotoxicity assay.

Mutation Research 607: 160-175.

Billinton N et al. (2008)

Interlaboratory assessment of the GreenScreen HC GADD45a-GFP genotoxicity screening assay: an enabling study for independent validation as an alternative method.

### Mutation Research in Press

TABLE 1
Validation of Vitotox, GreenScreen GC and RadarScreen Assays
against Ames tests (mutagenicity)

| | Vitotox | n | Radarscreen | n | Greenscreen | n |
|---|---|---|---|---|---|---|
| sensitivity | 0.90 | 48 | 0.55 | 47 | 0.39 | 33 |
| specificity | 0.90 | 108 | 0.52 | 107 | 0.98 | 48 |
| predictivity | 0.90 | 156 | 0.53 | 154 | 0.74 | 81 |

Reference Ames test: P; Gee, D M Maron, B N Ames, Detection and classification of mutagens: A set of base-specific Salmonella tester strains; Proc Natl Acad Sci USA (1994) 91, 11606-11610.

TABLE 2
Validation of Vitotox, GreenScreen GC and RadarScreen Assays
against in vitro Sister Chromatic Exchange (SCE) and Micronucleus Tests
(clastogenicity/aneuploidy)

| | Vitotox | n | Radarscreen | n | Greenscreen | n |
|---|---|---|---|---|---|---|
| sensitivity | 0.29 | 85 | 0.80 | 83 | 0.22 | 23 |
| specificity | 0.89 | 47 | 0.77 | 47 | 0.95 | 21 |
| predictivity | 0.51 | 132 | 0.78 | 130 | 0.57 | 44 |

The data of the different tests were compared with data present in the literature for the in vitro SCE or Micronucleus test. Sometimes comparison could only be made with one test. When results of both tests were available, and were different the compound was considered as being clastogenic.

Reference in vitro SCE: Huttner K M, Ruddle F H. Study of mitomycin C-induced chromosomal exchange. Chromosoma. 1976 Jun. 30; 56(1):1-13.

Reference Micronucleus test: Matter B E, Grauwiler J. Proceedings: The micronucleus test as a simple model, in vivo, for the evaluation of drug-induced chromosome aberrations. Comparative studies with 13 compounds. Mutat Res. 1975 August; 29(2):198-9.

TABLE 3
Validation of Vitotox and RadarScreen Assays for Genotoxicity Prediction

| | Sensitivity | | | | | |
|---|---|---|---|---|---|---|
| | | Muta- | | Clasto- | | Carcino- |
| | n | genicity | n | genicity | n | genicity |
| Vitotox | 43/48 | 0.90 | 25/85 | 0.29 | 15/50 | 0.30 |
| Radarscreen | 26/47 | 0.55 | 66/83 | 0.80 | 40/49 | 0.82 |
| Vitotox + | | | | | | |
| Radarscreen | 46/48 | 0.96 | 66/85 | 0.78 | 41/50 | 0.82 |

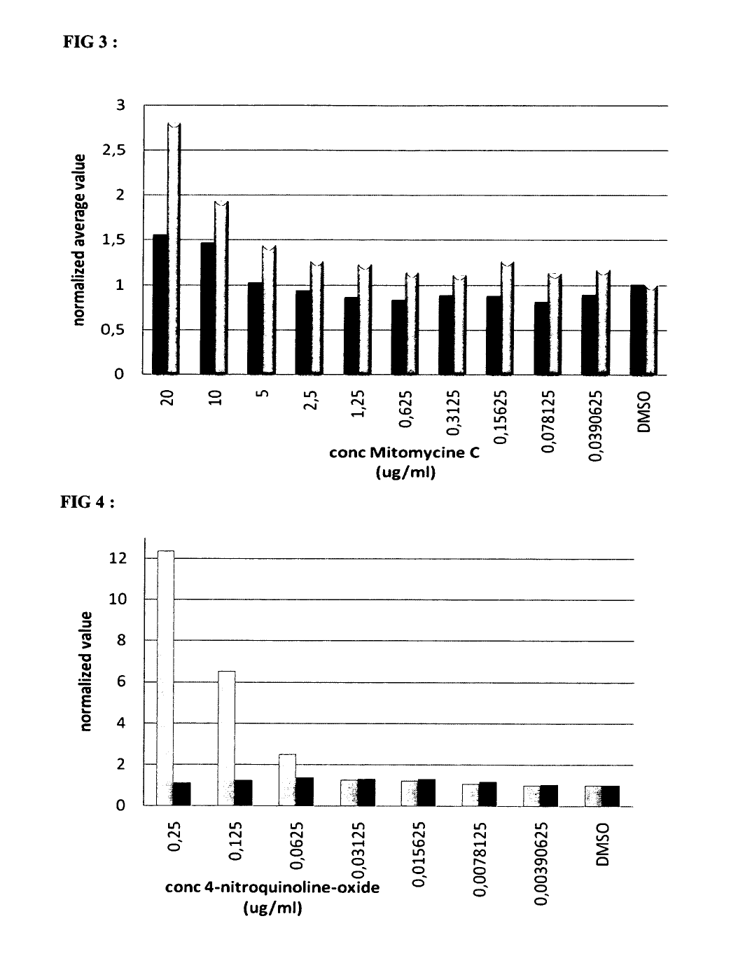

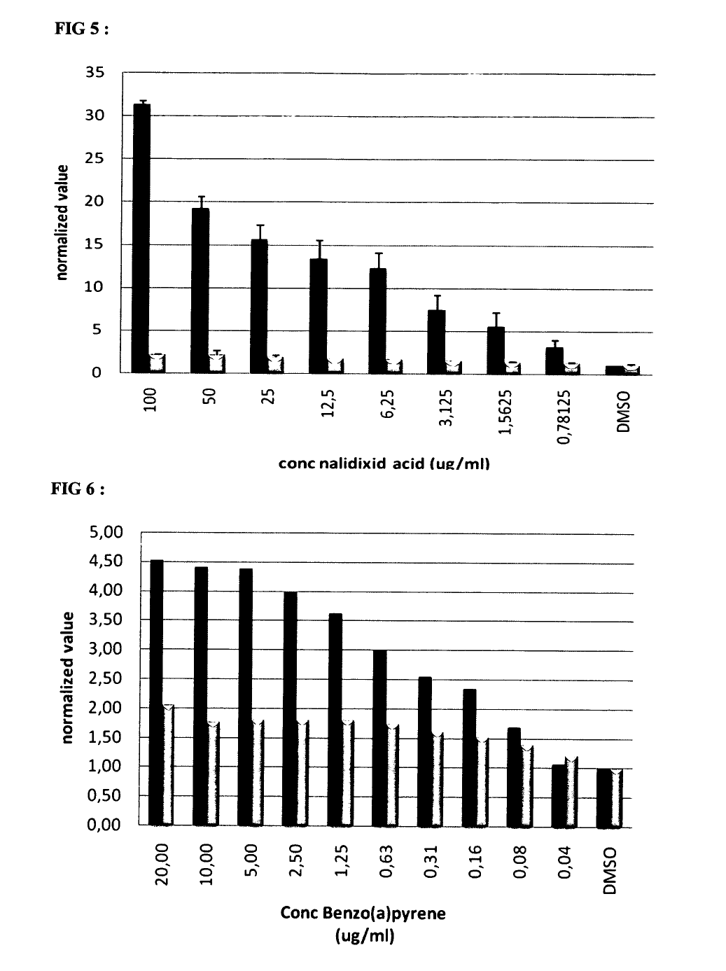

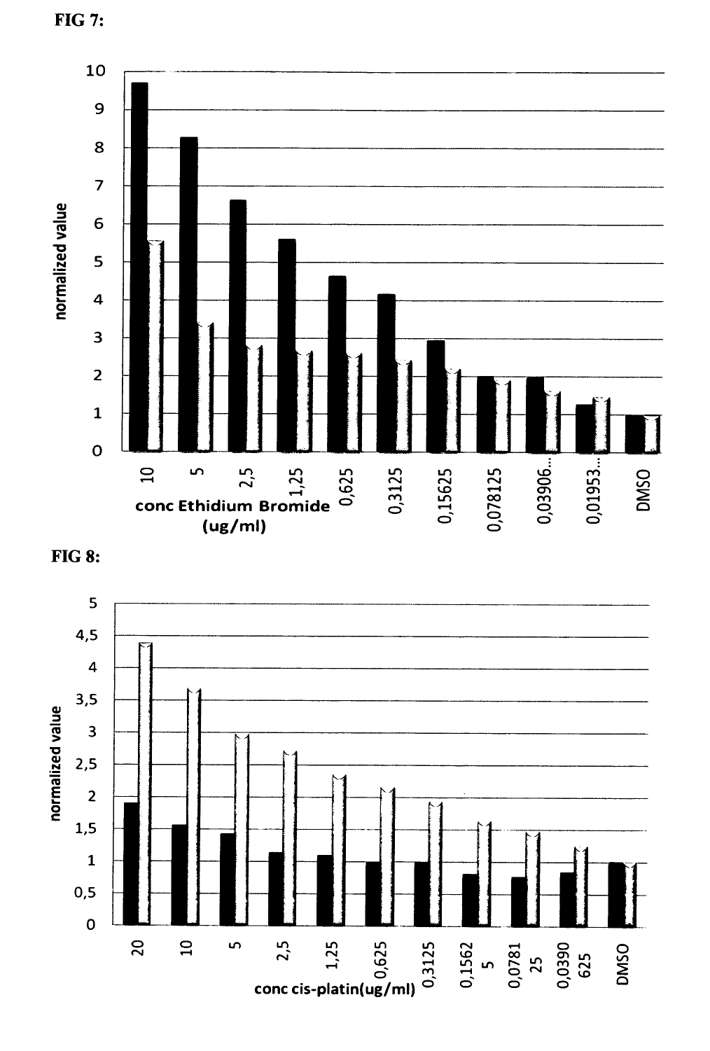

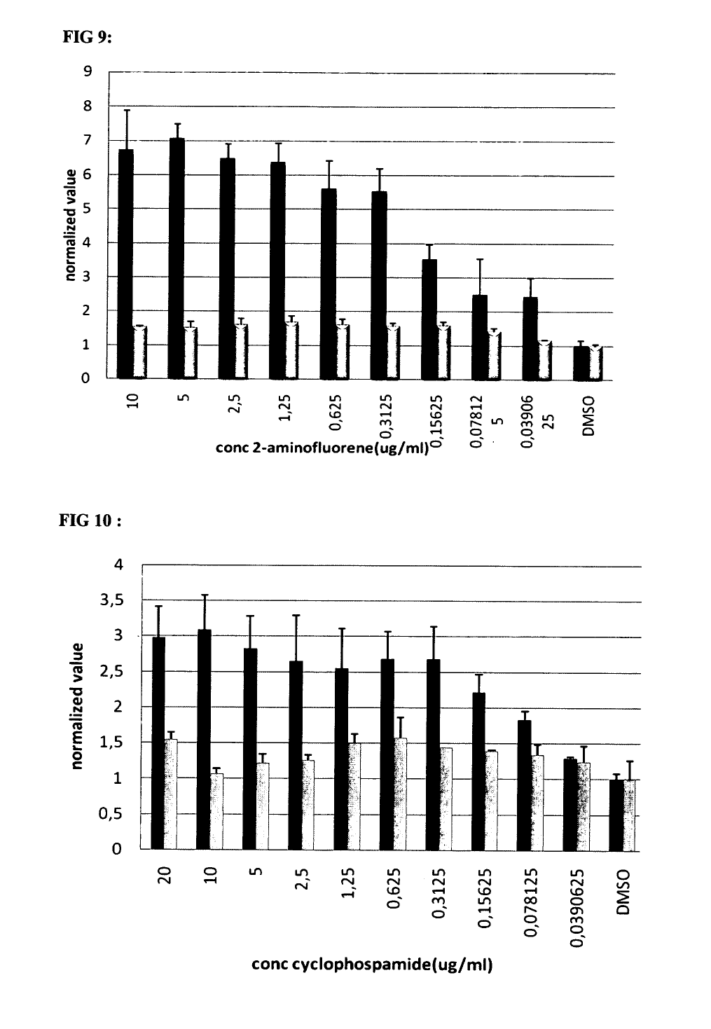

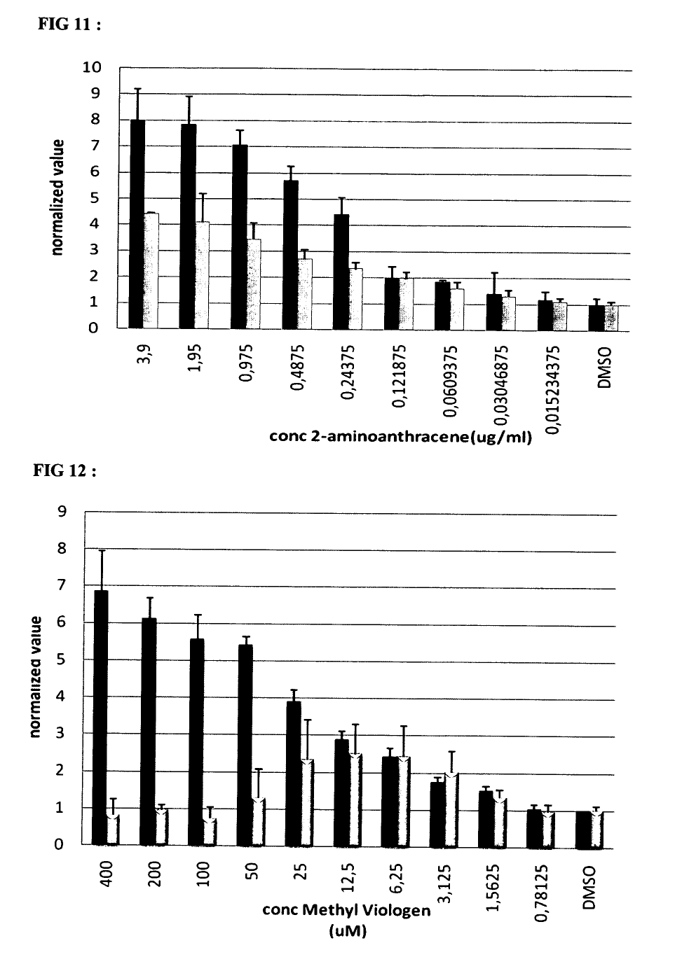

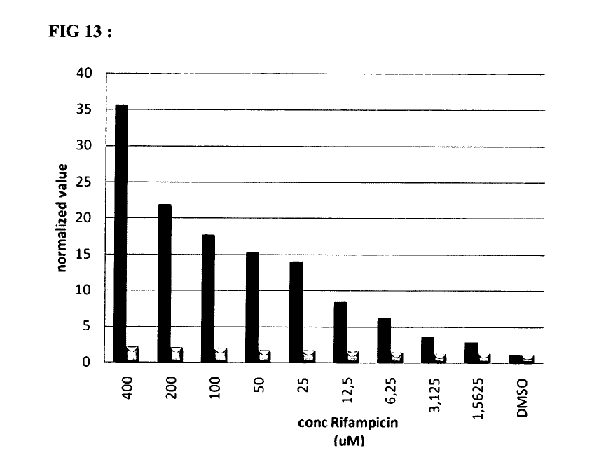

### Interpretation of Graphs of Reference Compounds (see FIG. 2-13)

For each compound the induction factor is set out in function of the concentration of the compound.

The induction factor is obtained as described above. Results from incubation with S9 as well as the results for incubation without S9 are given. Compounds that need metabolic activation (eg benzopyrene, ethidium bromide) will give a higher signal when analysed with S9 than when analysed without S9.

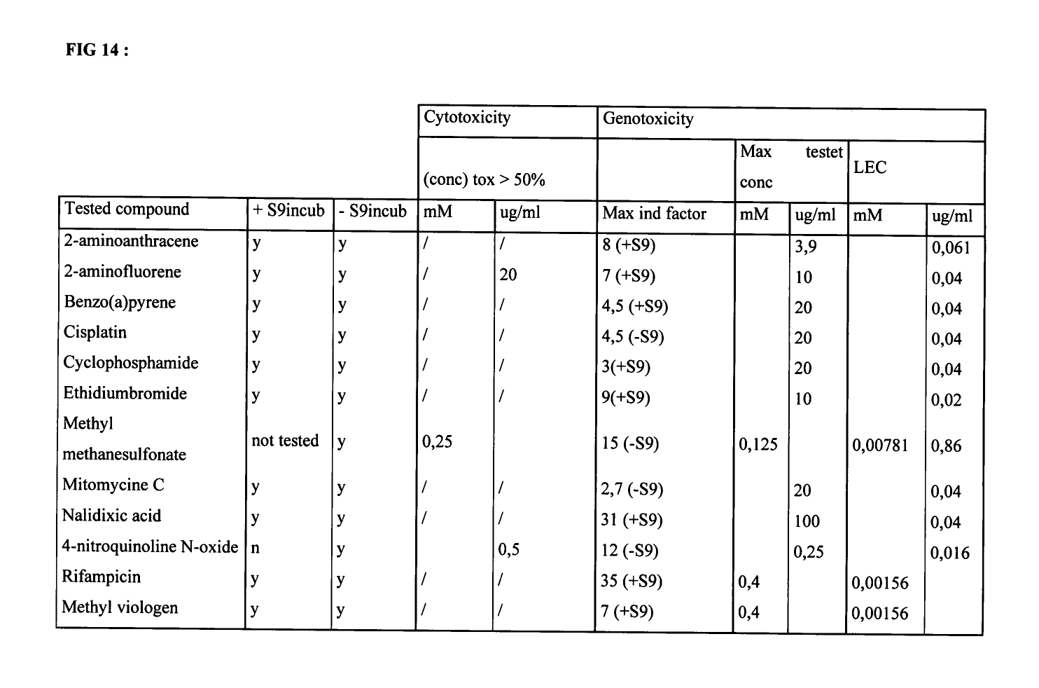

### Interpretation of Table 4 (see FIG. 14)

For each compound is given whether there is an induction with/without S9 and what is the highest induction factor with/without S9.

The highest test concentrations of the compounds are given.

Lowest concentrations of compounds with a cytotoxic effect are given. A compound is considered to be cytotoxic when yeast cell growth is reduced to 50% or less in comparison with the control DMSO.

The Lowest Effective Concentration (LEC) of the compound is the lowest concentration tested at which the induction factor is 1.5 or higher.

1. An eukaryotic cell characterized by:
the presence of a recombinant vector wherein the RAD54 regulatory element is operatively linked to a reporter protein
a defective base excision repair (BER) pathway; and
an impaired or reduced activity of export pumps.

2. The eukaryotic cell as claimed in claim 1 wherein the recombinant vector is integrated in the genome.

3. The eukaryotic cell as claimed in claim 1 wherein the export pumps are Snq2, Pdr5 and Yor1.

4. The eukaryotic cell as claimed in claim 1 wherein the reporter protein is the beta-galactosidase enzyme.

5. The eukaryotic cell as claimed in claim 1 wherein the recombinant vector is the vector of FIG. 1 or a functional derivative thereof.

6. The eukaryotic cell as claimed in claim 1 wherein the eukaryotic cell is a yeast cell comprising a SKAM4 cell, said SKAM4 cell;
comprising a recombinant vector sequence, wherein the RAD54 regulatory element is operatively linked to a reporter protein
having a defect in the BER DNA repair pathway caused by inactivation of MAG1; and
having impaired activity of the export pumps Snq2, Pdr5 and Yor1.

7. A method for preparing a cell as claimed in claim 1 comprising:
making the RAD54-reporter recombinant vector;
integrating the RAD54-reporter recombinant vector into the genome;
inactivating the BER pathway; and
deleting the export pumps.

8. A method comprising subjecting a cell according to claim 1 to an agent and monitoring the activity of the reporter protein, wherein an increase in reporter gene expression and/or reporter protein activity indicates that the agent causes or potentiates DNA damage.

9. The method as described in claim 8 comprising:
preparing yeast cells in the exponential growth phase;
adding the agent to be tested;
incubating the cells; and
monitoring the expression of the reporter gene or the activity of the reporter protein.

10. The method as claimed in claim 9 wherein the activity of the reporter protein is measured by adding a lysis buffer containing a beta-galactosidase substrate to the cell culture, wherein the substrate is directly or indirectly converted to a luminescent product.

11. The method as claimed in claim 10, wherein the beta-galactosidase substrate is cleaved by beta-galactosidase to luciferin and galactose and wherein luciferin is then used in a firefly luciferase reaction to generate light.

12. The method as claimed in claim 11, wherein the lysis buffer contains both the beta-galactosidase substrate and the firefly luciferase and the lysis of the cells and the monitoring of the reporter protein activity is performed in one step.

13. The method as claimed in claim 7 further comprising adding S9 extract to the cell culture prior to subjecting said cells to said agent.

14. A combination of a prokaryotic-based genotoxicity screening with a method as claimed in claim 7.

15. A kit comprising the cells as defined in claim 1.


SEQUENCE LISTING (11 sequences)

SEQ ID NO: 1 - DNA, 298 bp, Saccharomyces Cerevisiae
aagcttatgtatcaaaaatttaacatcttgaaaatacacaagtggtgcaaagatgtgtcacgttctggacctgagtggtgccatgtatgctatttaacatgcaaaggggaagacccttccgccttactgcaataataaaaagtattttacgcgttacccaatatagcaaagtttcgcgcaaaaaaaaaaataaaaaacaattacaaacaaaagaaaaaaaaggaaataatagaagatctaactgaagcgaaggccaaaactcttctcacttgacgtaatagccgatacaaaatctaga

SEQ ID NO: 2 - DNA, 3078 bp, Saccharomyces Cerevisiae
accatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataa

SEQ ID NO: 3 - protein, 1023 aa, Saccharomyces Cerevisiae
TMITDSLAVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQK

SEQ ID NO: 4 - DNA, 4506 bp, Saccharomyces Cerevisiae
atgagcaatatcaaaagcacgcaagatagctctcataatgctgtcgctagaagctcaagcgcttcttttgcagcttcagaagaatcatttacgggcataacccatgacaaagatgagcagagcgataccccggcggataaactaacaaaaatgctgacaggacctgcaagagacactgcgagccagattagtgccactgtgtctgaaatggcgccagatgtcgtatctaaagtggagtcatttgcagatgcactatcccgtcatacaacgagaagcggtgcctttaatatggattcagatagtgacgatgggttcgatgcccatgccatctttgaaagttttgtaagagacgctgatgagcaaggcatccatatccgcaaggctggtgttaccatagaggacgtaagcgctaaaggtgtggatgcgagtgccctagaaggtgctacctttggtaacattctttgtttaccgttgaccatctttaaaggtattaaggctaagaggcatcaaaagatgagacagatcataagcaatgtcaatgccctggcagaagcgggtgaaatgattttggttcttggaaggcctggtgctggttgttcctcctttttaaaagtaacagctggtgaaatagatcagtttgccggtggtgtttccggtgaagtagcatatgatggtattccccaagaagaaatgatgaaacgatataaagcagatgttatttacaatggtgagttggatgttcatttcccttatttaacagttaagcaaactttggatttcgctattgcctgcaaaacgcctgctctcagagtcaataacgtttccaaaaaggaatacattgcatccagaagagatttatatgcaaccattttcggtctaaggcatacctataataccaaagttggtaacgatttcgttagaggtgtatctggtggtgaacgtaagcgtgtttccattgccgaggctttggcagccaaaggttccatttactgttgggataatgccactagaggtttggatgcgtctacggccttagaatacgcaaaagccatccgtattatgacaaacttattgaaatcaaccgcttttgttacaatttatcaggcaagtgaaaacatttacgaaacatttgataaagtcactgtcctttattctggtaagcaaatttattttggtttgatccacgaggcaaaaccttatttcgcaaaaatgggttatttgtgtcctccaaggcaagcaacagctgaatttttaaccgcgttgactgatccaaatggattccatctgatcaagccaggttatgaaaataaagtaccaagaaccgctgaggaattcgaaacatattggttaaattctccagagtttgctcaaatgaaaaaagatatcgctgcttataaagagaaggtcaataccgaaaagactaaagaagtttatgacgaatcgatggctcaagagaaatccaaatatacgagaaagaagtcttattatacagtgtcatattgggaacaagttaaactgtgtacccaacgtgggttccaaagaatttacggtaacaagagttatacagtcatcaatgtctgctctgcaataattcaatcttttattactggatcattattttacaataccccttcatccacttccggtgctttttcaagaggtggtgtgttgtattttgcgctactatattattctttgatgggactggcgaatatttcttttgaacataggccaatcttacaaaagcacaagggctattctttgtatcatccttcagctgaggcaattggctccactctggcatctttccccttcagaatgattggtttgacctgtttctttatcattttattcttcctatctgggttgcacagaacagcgggatcattttttaccatctatttgttcttaaccatgtgttcagaggcgatcaatggtttatttgagatggtttcttcagtatgtgacactctttctcaagctaactctatctcgggtattctgatgatgtctatctcaatgtactctacctatatgatccaattgccttcgatgcatccatggtttaaatggatatcgtacgtactacctatcaggtacgccttcgagtcgatgttaaatgccgaatttcacggtaggcatatggattgtgctaacactctagtacccagtggaggagactatgataatttatccgatgactacaaagtatgtgcttttgttggttcgaaaccaggtcagtcttatgtgcttggtgatgactaccttaaaaatcaatttcagtacgtttataagcacacgtggagaaactttggtatcttgtggtgctttttactgggttatgttgttttgaaagtgatattcacagaatataagaggcctgtgaaaggtggtggtgatgctcttatcttcaagaaaggatcaaaaagatttatcgcacatgcagatgaagaatctccagacaatgtcaatgatatagatgccaaagagcaattctccagtgaaagtagcggcgcaaatgatgaagtatttgatgatttagaagccaaaggtgttttcatttggaaggacgtatgctttactattccatatgaaggcggtaagagaatgcttttggataatgtttcaggttattgtattccaggtaccatgacggccttgatgggagagtcaggtgctggtaaaacaactttgttaaatactcttgctcaaagaaatgtcggtatcattactggtgatatgcttgtcaatggacgtcccattgatgcgagtttcgaaaggcgtacaggttatgtacaacaacaggatatacatatcgcagagttaactgttagggaatcgttgcagttttctgctcgtatgcgtcgccctcagcatttgcctgattctgaaaaaatggattatgtggaaaaaatcatcagagttttgggaatggaagagtatgcggaagcccttgttggtgaggttggttgtggtttaaacgttgaacagagaaagaagctgtctattggtgttgaactagtcgccaaaccagacttattattattcctcgatgaacctacatcaggtttggattctcaatcttcatgggccattattcaattattaagaaagttatcaaaagctggccaatccattctttgtacgatccatcaaccttcagctactctgttcgaagagtttgatagattactacttttgaggaagggtggacaaactgtttatttcggagatattggtaagaactctgccaccattttgaactactttgaaaggaatggggcaagaaaatgtgattctagtgaaaatcctgctgaatatattttagaggctattggtgccggtgccacagcatccgtcaaagaagactggcacgaaaaatggttgaactctgtcgagtttgaacaaacaaaagaaaaagtacaggatttaataaatgatttatcgaaacaagaaactaaatccgaagttggagacaaaccttccaaatatgctacttcttatgcttaccagttcagatatgttttaatcagaacctctacttcattttggagaagtctgaattacatcatgtcaaagatgatgctaatgctggttggtggtctgtatattggtttcacatttttcaatgttggtaaaagttatgtcggcttacaaaatgcgatgttcgcggcatttatctctattatcttgtctgctcctgcaatgaaccaaatccaaggacgtgctattgcctccagagaactttttgaagttagggaatcccaatctaacatgtttcactggtcgctggtgttgatcactcagtacttgagcgaacttccctatcatttatttttttcgacaattttctttgtctcatcgtattttccattaagaatcttcttcgaagcgtcaagatctgcggtgtactttttgaattactgcattatgttccagttatactatgttggtcttggcttaatgatcctatatatgtcaccgaaccttccatccgctaatgttatcttaggtttgtgtctgtcatttatgctttctttctgtggtgttacacaacctgtctcattgatgcctggcttctggacattcatgtggaaggcttccccatacacatattttgttcagaatctggtcggaattatgctgcacaaaaaaccagtcgtatgcaaaaagaaagaactaaactacttcaacccaccaaacggctcaacgtgtggagagtacatgaaaccctttttggaaaaagctactggttacatcgaaaatcctgatgctacgtcagattgtgcatactgtatttacgaagttggagataattatttgacacatatcagctctaagtatagctacttgtggagaaattttggaatattttggatttacattttcttcaatatcattgctatggtttgtgtgtattacctcttccatgtaagacaatcttccttcctaagccccgtatctatactcaataaaattaaaaacataaggaaaaagaagcagtaa

SEQ ID NO: 5 - protein, 1501 aa, Saccharomyces Cerevisiae
MSNIKSTQDSSHNAVARSSSASFAASEESFTGITHDKDEQSDTPADKLTKMLTGPARDTASQISATVSEMAPDVVSKVESFADALSRHTTRSGAFNMDSDSDDGFDAHAIFESFVRDADEQGIHIRKAGVTIEDVSAKGVDASALEGATFGNILCLPLTIFKGIKAKRHQKMRQIISNVNALAEAGEMILVLGRPGAGCSSFLKVTAGEIDQFAGGVSGEVAYDGIPQEEMMKRYKADVIYNGELDVHFPYLTVKQTLDFAIACKTPALRVNNVSKKEYIASRRDLYATIFGLRHTYNTKVGNDFVRGVSGGERKRVSIAEALAAKGSIYCWDNATRGLDASTALEYAKAIRIMTNLLKSTAFVTIYQASENIYETFDKVTVLYSGKQIYFGLIHEAKPYFAKMGYLCPPRQATAEFLTALTDPNGFHLIKPGYENKVPRTAEEFETYWLNSPEFAQMKKDIAAYKEKVNTEKTKEVYDESMAQEKSKYTRKKSYYTVSYWEQVKLCTQRGFQRIYGNKSYTVINVCSAIIQSFITGSLFYNTPSSTSGAFSRGGVLYFALLYYSLMGLANISFEHRPILQKHKGYSLYHPSAEAIGSTLASFPFRMIGLTCFFIILFFLSGLHRTAGSFFTIYLFLTMCSEAINGLFEMVSSVCDTLSQANSISGILMMSISMYSTYMIQLPSMHPWFKWISYVLPIRYAFESMLNAEFHGRHMDCANTLVPSGGDYDNLSDDYKVCAFVGSKPGQSYVLGDDYLKNQFQYVYKHTWRNFGILWCFLLGYVVLKVIFTEYKRPVKGGGDALIFKKGSKRFIAHADEESPDNVNDIDAKEQFSSESSGANDEVFDDLEAKGVFIWKDVCFTIPYEGGKRMLLDNVSGYCIPGTMTALMGESGAGKTTLLNTLAQRNVGIITGDMLVNGRPIDASFERRTGYVQQQDIHIAELTVRESLQFSARMRRPQHLPDSEKMDYVEKIIRVLGMEEYAEALVGEVGCGLNVEQRKKLSIGVELVAKPDLLLFLDEPTSGLDSQSSWAIIQLLRKLSKAGQSILCTIHQPSATLFEEFDRLLLLRKGGQTVYFGDIGKNSATILNYFERNGARKCDSSENPAEYILEAIGAGATASVKEDWHEKWLNSVEFEQTKEKVQDLINDLSKQETKSEVGDKPSKYATSYAYQFRYVLIRTSTSFWRSLNYIMSKMMLMLVGGLYIGFTFFNVGKSYVGLQNAMFAAFISIILSAPAMNQIQGRAIASRELFEVRESQSNMFHWSLVLITQYLSELPYHLFFSTIFFVSSYFPLRIFFEASRSAVYFLNYCIMFQLYYVGLGLMILYMSPNLPSANVILGLCLSFMLSFCGVTQPVSLMPGFWTFMWKASPYTYFVQNLVGIMLHKKPVVCKKKELNYFNPPNGSTCGEYMKPFLEKATGYIENPDATSDCAYCIYEVGDNYLTHISSKYSYLWRNFGIFWIYIFFNIIAMVCVYYLFHVRQSSFLSPVSILNKIKNIRKKKQ

SEQ ID NO: 6 - DNA, 4536 bp, Saccharomyces Cerevisiae
atgcccgaggccaagcttaacaataacgtcaacgacgttactagctactcctccgcgtcttcttctactgaaaacgctgctgatctacacaattataatgggttcgatgagcatacagaagctcgaatccaaaaactggcaaggactctgaccgcacagagtatgcaaaactccactcaatcggcacccaacaaaagtgatgctcagtctatattttctagcggtgtggaaggtgtaaacccgatattctctgatcctgaagctccaggctatgacccaaaattggaccccaactccgaaaatttttctagtgccgcctgggttaagaatatggctcacctaagtgcggcagaccctgacttttataagccttattccttaggttgcgcttggaagaacttaagtgcttctggtgcttccgcagatgtcgcctatcagtcaactgtggttaatattccatacaaaatcctaaaaagtgggctgagaaagtttcaacgttctaaagaaaccaatactttccaaatcttgaaaccaatggatggttgcctaaacccaggtgaattgctagtcgttttaggtagaccaggctctggctgtactactttattaaaatccatctcttcaaatactcatggttttgatcttggtgcagatactaaaatttcttacagcggctactcaggtgatgatattaagaaacattttcgtggtgaagttgtttacaacgcagaagctgatgtacatctgcctcatttaacagtcttcgaaactttggttacagtagcgaggttgaaaaccccacagaaccgtatcaagggtgtcgatagggaaagttatgcgaatcatttggcggaagtagcaatggcaacgtacggtttatcgcatacaaggaatacaaaagttggtaacgacatcgtcagaggtgtttccggtggtgaaaggaagcgtgtctccattgctgaagtctccatctgtggatccaaatttcaatgctgggataatgctacaaggggtttggattccgctaccgctttggaatttattcgtgccttaaagactcaagctgatatttccaatacatctgccacagtggccatctatcaatgttctcaagatgcgtacgacttgttcaataaagtctgtgttttggatgatggttatcagatctactatggccccgccgataaggccaagaagtactttgaagatatggggtatgtttgtccaagcagacaaaccaccgcagattttttgacctcagttacaagtccctctgagagaaccctgaacaaagatatgctaaaaaaaggtattcatataccacagaccccgaaggaaatgaacgattactgggtaaaatctccaaattacaaagagctaatgaaagaagtcgaccaacgattattgaatgacgatgaagcaagccgtgaagctattaaggaagcccacattgctaagcagtccaagagagcaagaccttcctctccttatactgtcagctacatgatgcaagttaaatacctattaatcagaaatatgtggagactgcgaaataatatcgggtttacattatttatgattttgggtaactgtagtatggctttaatcttgggttcaatgtttttcaagatcatgaaaaagggtgatacttctacattctatttccgtggttctgctatgttttttgcaattctattcaatgcattttcttctctgttagaaatcttttcgttatatgaggccagaccaatcactgaaaaacatagaacatattcgttataccatccaagtgctgacgcttttgcatcagttctatcagaaataccctcaaagttaatcatcgctgtttgcttcaatataatcttctatttcttagtagactttagaagaaatggtggtgtattctttttctacttattaataaacattgtcgcggttttctccatgtctcacttgtttagatgtgttggttccttaacaaagacattgtcagaagctatggttcccgcttctatgttattgttggctctatccatgtataccggttttgctattcctaagaagaagatcctacgttggtctaaatggatttggtatatcaatccgttggcttacttattcgaatctttgttaattaacgagtttcatggtataaaattcccctgcgctgaatatgttcctcgtggtcctgcgtatgcaaacatttctagtacagaatctgtttgtaccgtggttggagctgttccaggccaagactatgttctgggtgatgatttcattagaggaacttatcaatactaccacaaagacaaatggcgtggtttcggtattggtatggcttatgtcgtcttctttttctttgtctatctattcttatgtgaatacaacgagggtgctaaacaaaaaggtgaaatattagttttcccacgcagtatagttaaaagaatgaagaaaagaggtgtactaactgaaaagaatgcaaatgaccccgaaaacgttggggaacgtagtgacttatccagcgataggaaaatgctacaagaaagctctgaagaggaatccgatacttacggagaaattggtttatccaagtcagaggctatatttcactggagaaacctttgttacgaagttcagattaaggccgaaacaagacgtattttgaacaatgttgatggttgggttaaaccaggtactttaacagctttaatgggtgcttcaggtgctggtaaaaccacacttctggattgtttggccgaaagggttaccatgggtgttataactggtgatatcttggtcaatggtattccccgtgataaatctttcccaagatccattggttattgtcagcaacaagatttgcatttgaaaactgccactgtgagggagtcattgagattttctgcttacctacgtcaaccagctgaagtttccattgaagaaaagaacagatatgttgaagaagttattaaaattcttgaaatggaaaaatatgctgatgctgttgttggtgttgctggtgaaggtttaaacgttgaacaaagaaaaagattaaccattggtgttgaattaactgccaaaccaaaactgttggtctttttagatgaacctacttctggtttggattctcaaactgcttggtctatttgtcagctaatgaaaaagttggcaaatcatggtcaagcaattctatgtactattcaccaaccctctgctattttgatgcaagaattcgatcgtttactatttatgcaacgtggtggtaagactgtctactttggcgacttgggcgaaggttgtaaaactatgatcgattattttgaaagccatggtgctcataaatgccctgctgacgccaacccagctgaatggatgctagaagttgttggtgcagctccaggctctcatgcaaatcaagattattacgaagtttggaggaattctgaagagtacagggccgttcaatctgaattagattggatggaaagagaattaccaaagaaaggttcgataactgcagctgaggacaaacacgaattttcacaatcaattatttatcaaacaaaattggtcagtattcgtctattccagcaatattggagatctccagattatttatggtcgaagtttattttaactattttcaatcaattgttcatcggtttcactttcttcaaagcaggaacctcgctacagggtttacaaaatcaaatgttggctgtgttcatgtttacggttattttcaatcctattctacaacaatacctaccatcttttgtccagcaaagagatttgtatgaggccagggaacgcccctcaaggactttttcttggatttcatttatcttcgctcaaatattcgtggaagttccatggaatatattggcaggtactattgcttattttatctactattatccaattggattttactccaacgcgtctgcagctggccagttgcatgaaaggggtgctttattttggttgttctcttgtgctttctacgtttatgttggttctatgggtctgcttgtcatttcattcaaccaagttgcagaaagtgcagctaacttagcctctttgttgtttacaatgtctttgtctttttgtggtgttatgactaccccaagtgccatgcctagattttggatattcatgtacagggtttcacctttgacttatttcattcaggctctgttggctgttggtgttgctaacgtagacgtcaaatgcgctgattacgaattgctagaattcacaccaccatccggtatgacatgtgggcagtacatggaaccatatttacaactagcaaagactggttacttaactgatgaaaatgccactgacacctgtagtttctgtcaaatatctacaaccaatgattacttagctaatgtcaattctttctacagtgagagatggagaaattatggtatcttcatctgttatattgcattcaattatatcgctggtgtctttttctactggttagcaagagtgcctaaaaagaacggtaaactctccaagaaataa

SEQ ID NO: 7 - protein, 1511 aa, Saccharomyces Cerevisiae
MPEAKLNNNVNDVTSYSSASSSTENAADLHNYNGFDEHTEARIQKLARTLTAQSMQNSTQSAPNKSDAQSIFSSGVEGVNPIFSDPEAPGYDPKLDPNSENFSSAAWVKNMAHLSAADPDFYKPYSLGCAWKNLSASGASADVAYQSTVVNIPYKILKSGLRKFQRSKETNTFQILKPMDGCLNPGELLVVLGRPGSGCTTLLKSISSNTHGFDLGADTKISYSGYSGDDIKKHFRGEVVYNAEADVHLPHLTVFETLVTVARLKTPQNRIKGVDRESYANHLAEVAMATYGLSHTRNTKVGNDIVRGVSGGERKRVSIAEVSICGSKFQCWDNATRGLDSATALEFIRALKTQADISNTSATVAIYQCSQDAYDLFNKVCVLDDGYQIYYGPADKAKKYFEDMGYVCPSRQTTADFLTSVTSPSERTLNKDMLKKGIHIPQTPKEMNDYWVKSPNYKELMKEVDQRLLNDDEASREAIKEAHIAKQSKRARPSSPYTVSYMMQVKYLLIRNMWRLRNNIGFTLFMILGNCSMALILGSMFFKIMKKGDTSTFYFRGSAMFFAILFNAFSSLLEIFSLYEARPITEKHRTYSLYHPSADAFASVLSEIPSKLIIAVCFNIIFYFLVDFRRNGGVFFFYLLINIVAVFSMSHLFRCVGSLTKTLSEAMVPASMLLLALSMYTGFAIPKKKILRWSKWIWYINPLAYLFESLLINEFHGIKFPCAEYVPRGPAYANISSTESVCTVVGAVPGQDYVLGDDFIRGTYQYYHKDKWRGFGIGMAYVVFFFFVYLFLCEYNEGAKQKGEILVFPRSIVKRMKKRGVLTEKNANDPENVGERSDLSSDRKMLQESSEEESDTYGEIGLSKSEAIFHWRNLCYEVQIKAETRRILNNVDGWVKPGTLTALMGASGAGKTTLLDCLAERVTMGVITGDILVNGIPRDKSFPRSIGYCQQQDLHLKTATVRESLRFSAYLRQPAEVSIEEKNRYVEEVIKILEMEKYADAVVGVAGEGLNVEQRKRLTIGVELTAKPKLLVFLDEPTSGLDSQTAWSICQLMKKLANHGQAILCTIHQPSAILMQEFDRLLFMQRGGKTVYFGDLGEGCKTMIDYFESHGAHKCPADANPAEWMLEVVGAAPGSHANQDYYEVWRNSEEYRAVQSELDWMERELPKKGSITAAEDKHEFSQSIIYQTKLVSIRLFQQYWRSPDYLWSKFILTIFNQLFIGFTFFKAGTSLQGLQNQMLAVFMFTVIFNPILQQYLPSFVQQRDLYEARERPSRTFSWISFIFAQIFVEVPWNILAGTIAYFIYYYPIGFYSNASAAGQLHERGALFWLFSCAFYVYVGSMGLLVISFNQVAESAANLASLLFTMSLSFCGVMTTPSAMPRFWIFMYRVSPLTYFIQALLAVGVANVDVKCADYELLEFTPPSGMTCGQYMEPYLQLAKTGYLTDENATDTCSFCQISTTNDYLANVNSFYSERWRNYGIFICYIAFNYIAGVFFYWLARVPKKNGKLSKK

SEQ ID NO: 8 - DNA, 6057 bp, Saccharomyces Cerevisiae
tctgcttctttgtttaatggtgtaagctgcctatatgttactattgagtactcatctcatcgcttctttcagaacaaaatttttcatattttttttttttccttttctttttttttttttctttgactgttacccggttgtttatatttgtaggaaaacaacaacgacagagaaaatatccttgcagtggcggctaatttgttagttgactgattgatcaccttcacttattaaagtaaaatcagcatacaagagatcagaagggagaaagagagtgggcaaggctatagtactttgaagaaagcatctttgaaccgaccagttctcttcacaagcaaaatctatatgactaaccgcaaggggcaaagggttgtgagagggcccgtctttctcccgctatagccgtcactggtatccctcctggctgcacaaatccgatagaaaggggaagaaggaagtttagtgccaccttatagcacgcagttactgtttacgctaaggagaggcatactcaatttttattagtcgccttctttagttgctgcgtttttatccacggttctctactaaatgcttgcgataagcgcttctattttcctccccaccgcgaggcggaaatggcacattttttttcttttgcttctgtgcttttgttgtaatttttggcatgtgctattgtatgaagataacgcgtggttccgtggaaatagccggaaattttgccgggaatatgacggacatgatttaacacccgtggaaatgaaaaaagccaaggtaagaaagtggcaatatttttcctacaaatagatctgctgtcccttaaatgattaccatacatatatatatttattacacatctgtcagaggtagctagcgaaggtgtcactgaaatattttttgttccagttagtataaatacggaggtagaacagctctccgcgtgtatatctttttttgcgctatacaagaacaggaagaacgcatttccatacctttttctccttacaggtgccctctgagtagtgtcacgaacgaggaaaaagattaatattactgtttttatattcaaaaagagtaaagccgttgctatatacgaatatgacgattaccgtgggggatgcagtttcggagacggagctggaaaacaaaagtcaaaacgtggtactatctcccaaggcatctgcttcttcagacataagcacagatgttgataaggacacatcgtcttcttgggatgacaaatctttgctgcctacaggtgaatatattgtggacagaaataagccccaaacctacttgaatagcgatgatatcgaaaaagtgacagaatctgatattttccctcagaaacgtctgttttcattcttgcactctaagaaaattccagaagtaccacaaaccgatgacgagaggaagatatatcctctgttccatacaaatattatctctaacatgtttttttggtgggttctacccatcctgcgagttggttataagagaacgatacagccgaacgatctcttcaaaatggatccgaggatgtctatagagaccctttatgacgactttgaaaaaaacatgatttactattttgagaagacgaggaaaaaataccgtaaaagacatccagaagcgacagaagaagaggttatggaaaatgccaaactacctaaacatacagttctgagagctttattattcacttttaagaaacagtacttcatgtcgatagtgtttgcaattctcgctaattgtacatccggttttaaccccatgattaccaagaggctaattgagtttgtcgaagaaaaggctatttttcatagcatgcatgttaacaaaggtattggttacgctattggtgcatgtttgatgatgttcgttaacgggttgacgttcaaccatttctttcatacatcccaactgactggtgtgcaagctaagtctattcttactaaagctgccatgaagaaaatgtttaatgcatctaattatgcgagacattgttttcctaacggtaaagtgacttcttttgtaacaacagatctcgctagaattgaatttgccttatcttttcagccgtttttggctgggttccctgcaattttggctatttgcattgttttattgatcgttaaccttggacccattgccttagttgggattggtatttttttcggtgggtttttcatatccttatttgcatttaagttaattctgggctttagaattgctgcgaacatcttcactgatgctagagttaccatgatgagagaagtgctgaataatataaaaatgattaaatattatacgtgggaggatgcgtatgaaaaaaatattcaagatattaggaccaaagagatttctaaagttagaaaaatgcaactatcaagaaatttcttgattgctatggccatgtctttgcctagtattgcttcattggtcactttccttgcaatgtacaaagttaataaaggaggcaggcaacctggtaatatttttgcctctttatctttatttcaggtcttgagtttgcaaatgtttttcttacctattgctattggtactggaattgacatgatcattggattgggccgtttgcaaagcttattggaggctccagaagatgatccaaatcagatgattgaaatgaagccttctcctggctttgatccaaaattggctttaaaaatgacacattgctcatttgagtgggaagattatgaattaaacgacgctattgaagaagcaaaaggagaagctaaagatgaaggtaaaaagaacaaaaaaaagcgtaaggatacatggggtaagccatctgcaagtactaataaggcgaaaagattggacaatatgttgaaagacagagacggcccggaagatttagaaaaaacttcgtttaggggtttcaaggacttgaacttcgatattaaaaagggcgaatttattatgattacgggacctattggtactggtaaatcttcattattgaatgcgatggcaggatcaatgagaaaaactgatggtaaggttgaagtcaacggggacttattaatgtgtggttatccatggattcaaaatgcatctgtaagagataacatcatattcggttcaccattcaataaagaaaagtatgatgaagtagttcgtgtttgctctttgaaagctgatctggatattttaccggcaggcgatatgaccgaaattggggaacgtggtattactttatctggtggtcaaaaggcacgtatcaatttagccaggtctgtttataagaagaaggatatttatctattcgacgatgtcctaagtgctgtcgattctcgtgttggtaaacacatcatggatgaatgtctaaccggaatgcttgctaataaaaccagaattttagcaacgcatcaattgtcactgattgagagagcttctagagtcatcgttttaggtactgatggccaagtcgatattggtactgttgatgagctaaaagctcgtaatcaaactttgataaatcttttacaattctcttctcaaaattcggagaaagaggatgaagaacaggaagcggttgttgccggtgaattgggacaactaaaatatgaatcagaggtaaaggaattgactgaactgaagaaaaaggctacagaaatgtcacaaactgcaaatagtggtaaaattgtagcggatggtcatactagtagtaaagaagaaagagcagtcaatagtatcagtctgaaaatataccgtgaatacattaaagctgcagtaggtaagtggggttttatcgcactaccgttgtatgcaattttagtcgttggaaccacattctgctcacttttttcttccgtttggttatcttactggactgagaataaattcaaaaacagaccacccagtttttatatgggtctttactccttctttgtgtttgctgctttcatattcatgaatggccagttcaccatactttgcgcaatgggtattatggcatcgaaatggttaaatttgagggctgtgaaaagaattttacacactccaatgtcatacatagataccacacctttgggacgtattctgaacagattcacaaaagatacagatagcttagataatgagttaaccgaaagtttacggttgatgacatctcaatttgctaatattgtaggtgtttgcgtcatgtgtattgtttacttgccgtggtttgctatcgcaattccgtttcttttggtcatctttgttctgattgctgatcattatcagagttctggtagagaaattaaaagacttgaagctgtgcaacggtcttttgtttacaataatttaaatgaagttttgggtgggatggatacaatcaaagcataccgaagtcaggaacgatttttggcgaaatcagattttttgatcaacaagatgaatgaggcgggataccttgtagttgtcctgcaaagatgggtaggtattttccttgatatggttgctatcgcatttgcactaattattacgttattgtgtgttacgagagcctttcctatttccgcggcttcagttggtgttttgttgacttatgtattacaattgcctggtctattaaataccattttaagggcaatgactcaaacagagaatgacatgaatagtgccgaaagattggtaacatatgcaactgaactaccactagaggcatcctatagaaagcccgaaatgacacctccagagtcatggccctcaatgggcgaaataatttttgaaaatgttgattttgcctatagacctggtttacctatagttttaaaaaatcttaacttgaatatcaagagtggggaaaaaattggtatctgtggtcgtacaggtgctggtaagtccactattatgagtgccctttacaggttgaatgaattgaccgcaggtaaaattttaattgacaatgttgatataagtcagctgggacttttcgatttaagaagaaaattagccatcattccacaagatccagtattatttaggggtacgattcgcaagaacttagatccatttaatgagcgtacagatgacgaattatgggatgcattggtgagaggtggtgctatcgccaaggatgacttgccggaagtgaaattgcaaaaacctgatgaaaatggtactcatggtaaaatgcataagttccatttagatcaagcagtggaagaagagggctccaatttctccttaggtgagagacaactattagcattaacaagggcattggtccgccaatcaaaaatattgattttggatgaggctacatcctcagtggactacgaaacggatggcaaaatccaaacacgtattgttgaggaatttggagattgtacaattttgtgtattgctcacagactgaagaccattgtaaattatgatcgtattcttgttttagagaagggtgaagtcgcagaattcgatacaccatggacgttgtttagtcaagaagatagtattttcagaagcatgtgttctagatctggtattgtggaaaatgatttcgagaacagaagttaatttatattatttgttgcatgatttttctcttttatttatttatatgttgccgatggtacaaattagtactagaaaagaaaacccactactatgacttgcagaaaaagttatgtgtgccatagatagatataattgcatacccacattgtatactcaaaattccgaaaagaacatttcattttttatgaggcaaactggacaacgccttcggtcctttttcattctagaaatatatatttatacatcattttcagaagatattcaaagaacttattgggatgtctgtttactgaataaagtatacacaaaatacgaatttaaaatggaaggcataaaatagaaaacttagaagtgaaaatcctaaaaccgaaggatatttcaaatacgtaaaagaagtgaaagataaaataaagcctaaataaggaagaaaagaagggataacatttttttttgttactttttgcttattcctcacctaaacagaaggaaaaagccattcttgtttaaagagtatttttaaagcgt

SEQ ID NO: 9 - protein, 1477 aa, Saccharomyces Cerevisiae
MTITVGDAVSETELENKSQNVVLSPKASASSDISTDVDKDTSSSWDDKSLLPTGEYIVDRNKPQTYLNSDDIEKVTESDIFPQKRLFSFLHSKKIPEVPQTDDERKIYPLFHTNIISNMFFWWVLPILRVGYKRTIQPNDLFKMDPRMSIETLYDDFEKNMIYYFEKTRKKYRKRHPEATEEEVMENAKLPKHTVLRALLFTFKKQYFMSIVFAILANCTSGFNPMITKRLIEFVEEKAIFHSMHVNKGIGYAIGACLMMFVNGLTFNHFFHTSQLTGVQAKSILTKAAMKKMFNASNYARHCFPNGKVTSFVTTDLARIEFALSFQPFLAGFPAILAICIVLLIVNLGPIALVGIGIFFGGFFISLFAFKLILGFRIAANIFTDARVTMMREVLNNIKMIKYYTWEDAYEKNIQDIRTKEISKVRKMQLSRNFLIAMAMSLPSIASLVTFLAMYKVNKGGRQPGNIFASLSLFQVLSLQMFFLPIAIGTGIDMIIGLGRLQSLLEAPEDDPNQMIEMKPSPGFDPKLALKMTHCSFEWEDYELNDAIEEAKGEAKDEGKKNKKKRKDTWGKPSASTNKAKRLDNMLKDRDGPEDLEKTSFRGFKDLNFDIKKGEFIMITGPIGTGKSSLLNAMAGSMRKTDGKVEVNGDLLMCGYPWIQNASVRDNIIFGSPFNKEKYDEVVRVCSLKADLDILPAGDMTEIGERGITLSGGQKARINLARSVYKKKDIYLFDDVLSAVDSRVGKHIMDECLTGMLANKTRILATHQLSLIERASRVIVLGTDGQVDIGTVDELKARNQTLINLLQFSSQNSEKEDEEQEAVVAGELGQLKYESEVKELTELKKKATEMSQTANSGKIVADGHTSSKEERAVNSISLKIYREYIKAAVGKWGFIALPLYAILVVGTTFCSLFSSVWLSYWTENKFKNRPPSFYMGLYSFFVFAAFIFMNGQFTILCAMGIMASKWLNLRAVKRILHTPMSYIDTTPLGRILNRFTKDTDSLDNELTESLRLMTSQFANIVGVCVMCIVYLPWFAIAIPFLLVIFVLIADHYQSSGREIKRLEAVQRSFVYNNLNEVLGGMDTIKAYRSQERFLAKSDFLINKMNEAGYLVVVLQRWVGIFLDMVAIAFALIITLLCVTRAFPISAASVGVLLTYVLQLPGLLNTILRAMTQTENDMNSAERLVTYATELPLEASYRKPEMTPPESWPSMGEIIFENVDFAYRPGLPIVLKNLNLNIKSGEKIGICGRTGAGKSTIMSALYRLNELTAGKILIDNVDISQLGLFDLRRKLAIIPQDPVLFRGTIRKNLDPFNERTDDELWDALVRGGAIAKDDLPEVKLQKPDENGTHGKMHKFHLDQAVEEEGSNFSLGERQLLALTRALVRQSKILILDEATSSVDYETDGKIQTRIVEEFGDCTILCIAHRLKTIVNYDRILVLEKGEVAEFDTPWTLFSQEDSIFRSMCSRSGIVENDFENRS

SEQ ID NO: 10 - DNA, 1219 bp, Saccharomyces Cerevisiae
gaattcttacataaagtttaagttatctatgaatcaatgagaattggccactgccctctgatatgacgatggaagtggtacttttcctttttaattttttactgagaatcaagagaagctagagagggattggtctcaatgaaactaaaaagggagtatgatgagttaataaaagcagacgctgttaaggaaatagcaaaagaattagggtctcgacctctagaggttgctcttcctgagaaatatattgctagacatgaagaaaagttcaatatggcttgcgaacacattttagagaaagatccatcactttttcccatacttaagaataatgaatttacgttgtacttgaaggagactcaagtccctaatacactcgaagattattttattaggcttgcaagcacaattttgtctcaacagatcagtggccaagcagctgaaagcatcaaggcaagggttgtcagtctttatggcggtgcatttcctgattacaaaatccttttcgaagacttcaaagacccagcaaaatgtgcagaaatcgcaaaatgtggattgagtaaaaggaaaatgatatatctagagtctcttgctgtctattttactgaaaaatataaggatatcgaaaagctcttcggtcaaaaagataatgatgaggaagtgattgaaagtttagttacgaatgtaaaaggtataggcccatggagtgccaaaatgttcttgatctccggattgaaaagaatggatgtatttgctcctgaagatctaggtattgctaggggtttttcaaaatacctttcagataagccagaattggaaaaagaattaatgcgtgaaagaaaagtagttaaaaagagtaagattaagcataagaaatacaactggaaaatatatgacgacgacataatggaaaaatgctctgaaacattttctccgtataggtctgtgtttatgttcatactttggaggctcgcgagcacaaatacagatgccatgatgaaggcagaagaaaatttcgtgaaatcctaacttaaagatatcatgtattactgacatttatataagaaaaaaaaaaataaaaataaatacaccctattgttgtgtgtgatgcctactaaagaaattcatatttacagcttctcgtagggataccaacatataataagaaaaagcctcacacatacaatccaaccattggagcgacaaagcggaaaagcga

SEQ ID NO: 11 - protein, 296 aa, Saccharomyces Cerevisiae
MKLKREYDELIKADAVKEIAKELGSRPLEVALPEKYIARHEEKFNMACEHILEKDPSLFPILKNNEFTLYLKETQVPNTLEDYFIRLASTILSQQISGQAAESIKARVVSLYGGAFPDYKILFEDFKDPAKCAEIAKCGLSKRKMIYLESLAVYFTEKYKDIEKLFGQKDNDEEVIESLVTNVKGIGPWSAKMFLISGLKRMDVFAPEDLGIARGFSKYLSDKPELEKELMRERKVVKKSKIKHKKYNWKIYDDDIMEKCSETFSPYRSVFMFILWRLASTNTDAMMKAEENFVKS